(19) United States
(12) Patent Application Publication
(10) Pub. No.: US 2006/0111442 A1
(43) Pub. Date: May 25, 2006

(54) Use of methyl pyruvate to increase cellular energy production downstream of glycolysis for the PARP-1 ablation of HIV without necrotic cell death caused by continuous, chronic PARP-1 activation through the concomitant depletion of ATP and NAD.

(75) Inventors: Stanley Charles Antosh, Palm Springs, CA (US); Anthony J. Meduri, New York City, NY (US)

Correspondence Address: OSMO, INC., 1271 VALDIVIA WAY, PALM SPRINGS, CA, 92262, US

(73) Assignees: Mr. Stanley Charles Antosh, Palm Springs, CA (US); Dr. Anthony J. Meduri, New York City, NY (US)

(21) Appl. No.: 10/904,648

(22) Filed: Nov. 20, 2004

(51) Int. Cl. A61K 31/22

(52) U.S. Cl. 514/546

(57) ABSTRACT

The present invention relates to the use of methyl pyruvic acid (a methyl ester of pyruvic acid) and/or methyl pyruvate (methyl pyruvate is the ionized form of methyl pyruvic acid) for the purpose of increasing cellular energy production thereby providing energy for the continuous activation of PARP-1 and up-regulation of PPAR. It is well known that chronic activation of PARP causes ATP and NAD depletion with concomitant cell death. PARP is known to prevent HIV replication by competitive receptor inhibition. Use of methyl pyruvate and/or methyl pyruvic acid can be effective when administered orally or infused on either a chronic and/or acute basis. In the following text, the terms “methyl pyruvate, methyl pyruvate compounds, methyl pyruvic acid” are used interchangeably.

Current U.S. Class: 435/194; 435/69.1; 435/183; 435/252.3; 435/254.11; 435/320.1; 530/350; 536/23.1; 536/23.2; 536/23.5

Intern'l Class: A01N 037/12; A61K 037/26; A61 K 031/198,70,19,22 C07D487/06; A61K31/55; A61P35/00; A61P35/28

Field of Search: 514/12,866; 435/69.1; 435/183; 435/194; 435/252.3; 435/254.11; 435/320.1; 530/300,324; 530/350; 536/23.1; 536/23.2; 536/23.5;

### REFERENCES CITED [REFERENCED BY]

U.S. Patent Documents

U.S. Pat. No. 5,045,454 September, 1991 Bertheussen 435/29.

U.S. Pat. No. 5,091,404 February, 1992 Elgebaly 514/401.

U.S. Pat. No. 5,192,762 March, 1993 Gray et al. 514/249.

U.S. Pat. No. 5,210,098 May, 1993 Nath 514/577.

U.S. Pat. No. 5,321,030June, 1994 Kaddurah-Daouk et al. 514/275.

U.S. Pat. No. 5,324,731 June, 1994 Kaddurah-Daouk et al. 514/275.

U.S. Pat. No. 5,741,661 Apr., 1998 Goldin et al. 435/29.

Foreign References:

EP0075805 1983-04 C07D 501/20 KYOWA HAKKO KOGYO CO., LTD Beta-lactam compound and a pharmaceutical composition containing the same

EP0233780 1987-08 C07D 501/36 ELI LILLY AND COMPANY O-substituted oximino cephalosporins

EP0370629 1990-05 C07C 251/60 IMPERIAL CHEMICAL INDUSTRIES PLC Fungicides

EP0400805 1990-12 C07D 501/20 Ishimaru, Toshiyasu Cephalosporin compounds and their use

EP0506149 1992-09 C07C 251/60 IMPERIAL CHEMICAL INDUSTRIES PLC Fungicides

EP0581187 1994-02 C07C 251/54 ONO PHARMACEUTICAL CO., LTD. Oxime derivatives

EP0708098 1996-04 C07D 277/34 SANKYO COMPANY LIMITED Oxime derivatives, their preparation and their therapeutic use

EP0916651 1999-05 C07C 259/02 Sankyo Company, Limited PHENYLALKYLCARBOXYLIC ACID DERIVATIVES

JP59167576 1983-03

JP62077391 1984-12

JP9323929 1996-04 JP11193272 1999-07

C07D 213/53 SANKYO CO LTD MEDICINE CONTAINING PHENYLALKYLCARBOXYLIC ACID DERIVATIVE WO9602507 1996-02 C07D 215/14 ABBOTT LABORATORIES IMINOXYCARBOXYLATES AND DERIVATIVES AS INHIBITORS OF LEUKOTRIENE BIOSYNTHESIS

WO9633724 1996-10 A61K 31/557 EVANS, Ronald, M. SELECTIVE MODULATORS OF PEROXISOME PROLIFERATOR ACTIVATED RECEPTOR-GAMMA, AND METHODS FOR THE USE THEREOF

WO9638427 1996-12 C07D 263/44 FUJIMOTO, Koichi AROMATIC OXYIMINO DERIVATIVES

WO9640128 1996-12 A61K 31/425 THE SALK INSTITUTE FOR BIOLOGICAL STUDIES MODULATORS OF PEROXISOME PROLIFERATOR ACTIVATED RECEPTOR-GAMMA, AND METHODS FOR THE USE THEREOF

WO9725042 1997-07 A61K 31/42 SMITH, STEPHEN, ALISTAIR USE OF AN ANTAGONIST OF PPAR-ALPHA AND PPAR-GAMMA FOR THE TREATMENT OF SYNDROM X

WO9731907 1997-09 C07D 263/56 BOSWELL, GRADY, EVAN SUBSTITUTED 4-HYDROXY-PHENYLALCANOIC ACID DERIVATIVES WITH AGONIST ACTIVITY TO PPAR-GAMMA

WO9805331 1998-02 A61K 31/45 LIGAND PHARMACEUTICALS INCORPORATED PREVENTION OR TREATMENT OF TYPE 2 DIABETES OR CARDIOVASCULAR DISEASE WITH PPAR MODULATORS

WO9904815 1999-02 A61K45/00 HASHIMOTO, SEIICHI MEDICINAL COMPOSITIONS WITH CHOLESTEROL-LOWERING EFFECT

WO9958510A1 issued 1999-Nov.-18 OXYIMINOALKANOIC ACID DERIVATIVES WITH HYPOGLYCEMIC AND HYPOLIPIDEMIC ACTIVITY

Other References
Rudinger, Peptide Hormones (Jun. 1976: J. Parsons Ed.) p. 1-6.*
Goodman & Gilman's, “The Pharmcological Basis of Therapeutics”, 6th Ed. (Macmillan Pub.:1980) pp. 5-10.*
Chemical Abstracts AN 1 982:49622, Bertolini et al., January 1981.*
Chemical Abstracts AN 89:82648, Rees et al, U.S. Pat. No. 4,871,774, Mar. 10, 1989.*
Annesley, T. and Walker, J., “Formation and Utilization of Novel High Energy Phosphate Reservoirs in Ehrlich Ascites Tumor Cells,” J. Biol. Chem., vol. 253, No. 22, 8120-5 (1978).
Beal, M., “Aging, Energy, and Oxidative Stress in Neurodegenerative Diseases,” Ann. Neurol., vol. 38, 357-66 (1995).
Beal, M., “Does Impairment of Energy Metabolism Result in Excitotoxic Neuronal Death in Neurodegenerative Illnesses?” Ann. Neurol., vol. 31, 119-30 (1992).
Beal, M. et al., “Age-Dependent Striatal Excitotoxic Lesions Produced by the Endogenous Mitochondrial Inhibitor Malonate,” J. Neurochem., vol. 61, 1147-50 (1993).
Brouillet, E. et al., “Chronic Mitochondrial Energy Impairment Produces Selective Striatal Degeneration and Abnormal Choreiform Movements in Primates,” PNAS, vol. 92, 7105-9 (1995).
Browne, S. et al., “Oxidative Damage and Metabolic Dysfunction in Huntington's Disease: Selective Vulnerability of the Basal Ganglia,” Ann. Neurol., vol. 41, 646-53 (1997).
Burbaeva, GSh et al., “Decreased Level of Immunoreactive Phosphokinase BB Isoenzymes in the Brain of Patients with Schizophrenia and Senile Dementia of the Alzheimer Type,” Zh. Nevropatol. Psikhiatr Im S S Korsakova, vol. 90, No. 7, 85-7 (1990)—abstract attached.
De Leon, M. et al., “Identification of Transcriptionally Regulated Genes After Sciatic Nerve Injury,” J. Neurosci. Res., vol. 29, 437-48 (1991).
Erecinska, M. and Silver, I., “ATP and Brain Function,” J. Cerebr. Blood Flow and Metabolism, vol. 9, 2-19 (1989).
Gu, M. et al:, “Mitochondrial Defect in Huntington's Disease Caudate Nucleus,” Ann. Neurol., vol. 39, 385-9 (1996).
Gurney, M. et al., “Motor Neuron Degeneration in Mice That Express a Human Cu,Zn Superoxide Dismutase Mutation,” Science, vol. 264, 1772-5 (1994).
Henshaw, R. et al., “Malonate Produces Striatal Lesions by Indirect NMDA Receptor Activation,” Brain Research, vol. 647, 161-6 (1994).
Hertz, L. and Peng, L., “Energy Metabolism at the Cellular Level of the CNS,” Can. J. Physiol. Pharmacol., vol. 70, S145-57 (1992).
Ito, M., “The Cellular Basis of Cerebellar Plasticity,” Corr. Opin. Neurobiol., vol. 1, 616-20 (1991).
Jenkins, B. et al., “Evidence for Impairment of Energy Metabolism in Vivo in Huntington's Disease Using Localized .sup.1 H NMR Spectroscopy,” Neurology, vol. 43, 2689-95 (1993).
Maker, H. et al., “Regional Changes in Cerebellar Creatine Phosphate Metabolism During Late Maturation,” Exp. Neurol., vol. 38, 295-300 (1973).
Manos, P. et al., “Creatine Kinase Activity in Postnatal Rat Brain Development and in Cultured Neurons, Astrocytes, and Oligodendrocytes,” J. Neurochem., vol. 56, 2101-7 (1991).
Molloy, G. et al., “Rat Brain Creatine Kinase Messenger RNA Levels are High in Primary Cultures of Brain Astrocytes and Oligodencrocytes and Low in Neurons,” J. Neurochem., vol. 59,1925-32 (1992).
Newman, E., “Regulation of Potassium Levels by Glial Cells in the Retina,” Trends NeurosciencI, vol. 8, 156-9 (1985).
Oblinger, M. et al., “Cytotypic Differences in the Protein Composition of the Axonally Transported Cytoskeleton in Mammalian Neurons,” J. Neurol., vol. 7, No. 2, 453-62 (1987).
Orlovskaia, D. D. et al., “Neuromorphology and Neurochemistry of Senile Dementias in the Light of Studies on Glial Response,” Vestn Ross Akad Med Nauk., vol. 8, 34-9 (1992)—abstract only.
Reichenbach, A., “Glial K+ Permeability and CNS K+ Clearance by Diffusion and Spatial Buffering,” Acad. Sci. New York, 272-86 (1991). Chemical Society, vol. 93, 5542-51(1971).
Schiffmann, R. et al., “Childhood Ataxia with Diffuse Central Nervous System Hypomyelination,” Ann. Neurol., vol. 35, 331-40 (1994).
Schultz, J. et al., “Blockade of Neuronal Nitric Oxide Synthase Protects Against Excitotoxicity in vivo,” J. Neurosci., vol. 15, No. 12, 8419-29 (1995).
Schultz, J. et al., “Inhibition of Neuronal Nitric Oxide Synthase by 7-Nitroindazole Protects Against MPTP-lnduced Neurotoxicity in Mice,” J. Neurochem., vol. 64, 936-9 (1995).
Stadhouders, A., et al., “Mitochondrial Creatine Kinase: A Major Constituent of Pathological Inclusions Seen in Mitochondrial Myopathies,” PNAS, vol. 91, No. 11, 5089-93 (1994).
Wang, T., “Synthesis and Properties of N-Acetimidoyl Derivatives of Glycine and Sarcosine,” JOC, vol. 39, No. 24, 3591-4 (1974).
Beal, M. F., et al, “Do defects in mitochondrial energy metabolism underlie the pathology of neurodegenerative diseases?” Trends Neurosci. 16: 125-131(1993).
Folbergrova, J., et al., “Focal and perifocal changes in tissue energy state during middle cerebral artery occlusion in normo- and hyperglycemic rats,” J. Cereb. Blood Flow Metab. 12: 25-33 (1992).
Ginsberg, M. D. and Busto, R., “Rodent models of cerebrasl ischemia (Progress Review),” Stroke 20:1627-1640 (1989).
Mosinger, J. L. and Olney, J. W., “Photothrombosis-induced ischemic neuronal degeneration in the rat retina,” Exp. Neurol. 105: 110-11 3, (1989).
J. W., “Excitatory amino acids and neuropsychiatric disorders,” Biol. Psychiatry 26:505-525 (1989).
Olmey. J. W., “NMDA antagonist neurotoxicity: Mechanism and prevention,” Science 254: 1515-1518 (1991).
Siesjo, B. K., Brain Energy Metabolism (John Wiley & Sons, New York, 1978)
Simon, R. P., et al., “Blockade of N-methyl-D-aspartate receptors may protect against ischemic damage in the brain,” Science 226: 850-852 (1984).
Voll, C. L. and Auer, R. N. “Insulin attenuates ischemic brain damage independent of its hypoglycemic effect,” J. Cereb. Blood Flow and Metabolism 11: 1006-1014 (1991).
Choi, D. W. At the scene of ischemic brain injury: is PARP a perp? Nat. Med. 3:1073-1074, 1997.
Jeggo, P. A. DNA repair: PARP-another guardian angel? Curr. Biol., 8: R49-R51, 1998.
Pieper, A. A., Verma, A., Zhang, J., and Snyder, S. H. Poly(ADP-ribose) polymerase, nitric oxide and cell death. Trends Pharmacol. Sci., 20: 1 71-181, 1999.
D'Amours, D., Desnoyers, S., D'Silva, I., and Poirier, G. G. Poly(ADP-ribosyl)ation reactions in the regulation of nuclear functions. Biochem. J., 342: 249-268, 1999. Mendoza-Alvarez, H., and Alvarez-Gonzalez, R. Poly(ADP-ribose) polymerase is a catalytic dimer and the automodification reaction is intermolecular. J. Biol. Chem., 268: 22575-22580, 1993.
Burkart, V., Wang, Z. Q., Radons, J., Heller, B., Herceg, Z., Stingl, L., Wagner, E. F., and Kolb, H. Mice lacking the poly(ADP-ribose) polymerase gene are resistant to pancreatic b-cell destruction and diabetes development induced by streptozocin. Nat. Med., 5: 314-319, 1999.
Masutani, M., Nozaki, T., Nakamoto, K., Nakagama, H., Suzuki, H., Kusuoka, O., Tsutsumi, M., and Sugimura, T. The response of Parp knockout mice against DNA damaging agents. Mutat. Res., 462: 159-166, 2000.
Masutani, M., Suzuki, H., Kamada, N., Watanabe, M., Ueda, O., Nozaki, T., Jishage, K., Watanabe, T., Sugimoto, T., Nakagama, H., Ochiya, T., and Sugimura, T. Poly(ADP-ribose) polymerase gene disruption conferred mice resistant to streptozo-tocin-induced diabetes. Proc. Natl. Acad. Sci. USA, 96: 2301-2304, 1999.
Lindahl, T., Satoh, M. S., Poirier, G. G., and Klungland, A. Post-translational modification of poly(ADP-ribose) polymerase induced by DNA strand breaks. Trends Biochem. Sci., 20: 405-411, 1995.
Decker, P., Miranda, E. A., de Murcia, G., and Muller, S. An improved nonisotopic test to screen a large series of new inhibitor molecules of poly(ADP-ribose) polymerase activity for therapeutic applications. Clin. Cancer Res., 5: 1169-1172, 1999.
Holl, V., Coelho, D., Weltin, D., Hyun, J. W., Dufour, P., and Bischoff, P. Modulation of the antiproliferative activity of anticancer drugs in hematopoietic tumor cell lines by the poly(ADP-ribose) polymerase inhibitor 6(5H)-phenanthridinone. Anticancer Res., 20: 3233-3241, 2000.
Szabo, C., and Dawson, V. L. Role of poly(ADP-ribose) synthetase in inflammation and ischaemia-reperfusion. Trends Pharmacol. Sci., 19: 287-298, 1998.
Takahashi, K., Pieper, A. A., Croul, S. E., Zhang, J., Snyder, S. H., and Greenberg, J. H. Post-treatment with an inhibitor of poly(ADP-ribose) polymerase attenuates cerebral damage in focal ischemia. Brain Res., 829: 46-54, 1999.
Shall, S., and de Murcia, G. Poly(ADP-ribose) polymerase-1: what have we learned from the deficient mouse model? Mutat. Res., 460: 1-15, 2000.
Jacobson, M. K., and Jacobson, E. L. Discovering new ADP-ribose polymer cycles: protecting the genome and more. Trends Biochem. Sci., 24: 415-417, 1999.
Ame, J. C., Rolli, V., Schreiber, V., Niedergang, C., Apiou, F., Decker, P., Muller, S., Hoger, T., Menissier-de Murcia, J., and de Murcia, G. PARP-2, a novel mammalian DNA damage-dependent poly(ADP-ribose) polymerase. J. Biol. Chem., 274: 17860-17868, 1999.
Johansson, M. A human poly(ADP-ribose) polymerase gene family (ADPRTL): cDNA cloning of two novel poly(ADP-ribose) polymerase homologues. Genomics, 57: 442-445, 1999.
Kickhoefer, V. A., Siva, A. C., Kedersha, N. L., Inman, E. M., Ruland, C., Streuli, M., and Rome, L. H. The 193-kD vault protein, VPARP, is a novel poly(ADP-ribose) polymerase. J. Cell Biol., 146: 91 7-928, 1999.
Sallmann, F. R., Vodenicharov, M. D., Wang, Z. Q., and Poirier, G. G. Characterization of sPARP-1. An alternative product of PARP-1 gene with poly(ADP-ribose) polymerase activity independent of DNA strand breaks. J. Biol. Chem., 275: 15504-15511, 2000.
Smith, S., Giriat, I., Schmitt, A., and de Lange, T. Tankyrase, a poly(ADP-ribose) polymerase at human telomeres. Science (Wash. DC), 282: 1484-1487, 1998. d'Adda di Fagagna, F., Hande, M. P., Tong, W. M., Lansdorp, P. M., Wang, Z. Q., and Jackson, S. P. Functions of poly(ADP-ribose) polymerase in controlling telomere length and chromosomal stability. Nat. Genet., 23: 76-80, 1999. Kong, L. B., Siva, A. C., Rome, L. H., and Stewart, P. L. Structure of the vault, a ubiquitous cellular component. Structure Fold Des., 7: 371-379, 1999. Pennisi, E. A possible new partner for telomerase. Science (Wash. DC), 282: 1395-1396, 1999.
Smith, S., and de Lange, T. Tankyrase promotes telomere elongation in human cells. Curr. Biol., 10: 1299-1302, 2000.
Kickhoefer, V. A., Rajavel, K. S., Scheffer, G. L., Dalton, W. S., Scheper, R. J., and Rome, L. H. Vaults are up-regulated in multidrug-resistant cancer cell lines. J. Biol. Chem., 273: 8971-8974, 1998.
Schroeijers, A. B., Siva, A. C., Scheffer, G. L., de Jong, M. C., Bolick, S. C., Dukers, D. F., Slootstra, J. W., Meloen, R. H., Wiemer, E., Kickhoefer, V. A., Rome, L. H., and Scheper, R. J. The M r 193,000 vault protein is up-regulated in multidrug-resistant cancer cell lines. Cancer Res., 60: 1104-1110, 2000.
Ame, J. C., Jacobson, E. L., and Jacobson, M. K. Molecular heterogeneity and regulation of poly(ADP-ribose) glycohydrolase. Mol. Cell Biochem., 1 93: 75-81, 1999. Lin, W., Ame, J. C., Aboul-Ela, N., Jacobson, E. L., and Jacobson, M. K. Isolation and characterization of the cDNA encoding bovine poly(ADP-ribose) glycohydrolase. J. Biol. Chem., 272: 11895-11901, 1997.

### FIELD OF THE INVENTION

The present invention relates to the field of immunology, more specifically viral immunology with a focus on HIV. The present invention further relates to ensuring genomic integrity and prevention of necrosis by ischemic events. The present invention even further relates to the use of methyl pyruvate for the purpose of increasing cellular energy production downstream of the glycolytic blockade induced by continuous PARP-1 activation. Providing ATP enables continuous, chronic activation of PARP-1. It is well known that chronic activation of PARP causes ATP and NAD depletion with concomitant necrotic cell death. PARP is known to prevent HIV replication by competitive receptor inhibition.

More particularly the present invention relates to enhancing the production of energy by utilizing methyl pyruvate which modulate the system for the purpose of increasing cellular energy production where the energy demand is ceaseless or energy metabolism is suppressed or defective. In the following text, the terms “methyl pyruvate, methyl pyruvate compounds, methyl pyruvic acid” are used interchangeably.

### BACKGROUND OF THE INVENTION

It is the object of the present invention to increase cellular energy production with the addition of Methyl Pyruvate and its supra-normal stimulation of the Krebs Cycle (TCA) to support the ATP and NAD requirements of PARP 1 activation. PARP-1 activation ensures genomic integrity and ablation of viral and more specifically HIV replication through competitive receptor inhibition. Additional aspects of this invention include prevention of necrosis by ischemic events as well as PPAR activation. The treatment of HIV infection with combinations of Nucleoside Reverse Transcriptase Inhibitors (NRTIs), highly active antiretroviral therapy (HAART) and Protease Inhibitors (P is) has been long accepted as the only efficacious treatment. However, there are myriad side effects in HIV infected patients who are treated with these drugs. The adage “show me a drug with no side-effects and I will show you a drug that does not work” is indeed valid with mention of only some of the side-effects below, merely the undesired expression of the drugs, the treatment, not the HIV infection. In the following text you will come to understand the value of Methyl Pyruvate in support of PARP-1 and up-regulation of PPAR.

Protease inhibitors decrease the viral load in HIV patients; however the patients develop hypertriglyceridemia, hypercholesterolemia, hypercortisolism and atherosclerosis. HIV-1 protease-inhibitor treatments are associated with a syndrome of peripheral lipodystrophy, central adiposity, breast hypertrophy in women, and hyperlipidaemia. HIV-associated lipodystrophy is a medical condition characterized by gradual changes in the distribution of body fat. The body fat located in the extremities and face disappears while body fat around the abdomen and upper back increases. Certain biochemical changes occur in association with these changes in fat distribution. Lipid levels particularly serum triglycerides are increased. HDL, the “good cholesterol” is decreased. Higher then normal level of insulin or insulin resistance is also found in this condition. This latter condition is one of the hallmarks of Type II diabetes.

Additional patient side effects of these treatments include lose of subcutaneous fat and metabolic abnormalities of reduced adiponectin levels, which may be related to disrupted subcutaneous adipogenesis and altered peroxisome proliferator-activated receptor-gamma transcription. Specifically, HIV protease inhibitors promote atherosclerotic lesion formation independent of dyslipidemia. Metabolic disorders in HIV-infected patients, especially those receiving highly active antiretroviral therapy (HAART) regimens containing protease inhibitors, are associated with insulin resistance as well as HAART-associated lipodystrophy. These metabolic disorders also include fat redistribution, diabetes, and hypertriglyceridemia. Insulin resistance alone, which is thought to play a central role in DM-2 induces an associated metabolic syndrome characterized by central obesity, hypertension, dyslipidemia and hypercoagulability.

Much debate currently exists regarding the contribution of NRTIs (Nucleoside Reverse Transcriptase Inhibitors) and P is to the development of HIV-associated lipodystrophy, with evidence the cytotoxicity exerted by NRTIs and P is occur via distinct mechanisms. NRTIs have the intrinsic ability to inhibit mitochondrial DNA (mtDNA) replication and P is have been demonstrated to inhibit adipocyte differentiation. However, there also appears to be distinct mechanisms of toxicity within each class. HIV-1 protease-inhibitors therapy is associated with increased levels of triglycerides, LDL-cholesterol and Lp(a). HIV-1 protease-inhibitors therapy is also responsible for the development of a lipodystrophy syndrome (insulin resistance), many data indicate that HIV-1 protease-inhibitors therapy itself modifies significantly lipid metabolism. Thus, it is obvious that alternative or adjunctive therapy is needed for persons infected with HIV.

Cells require energy to survive and perform their physiological functions, and it is generally recognized that the source of energy for cells is the glucose and oxygen delivered by the blood. There are two major components to the process by which cells utilize glucose and oxygen to produce energy. The first component entails anaerobic conversion of glucose to pyruvate, which releases a small amount of energy, and the second entails oxidative conversion of pyruvate to carbon dioxide and water with the release of a large amount of energy. Pyruvate is continuously manufactured in the living organism from glucose. The process by which glucose is converted to pyruvate involves a series of enzymatic reactions that occur anaerobically (in the absence of oxygen). This process is called “glycolysis”. A small amount of energy is generated in the glycolytic conversion of glucose to pyruvate, but a much larger amount of energy is generated in a subsequent more complicated series of reactions in which pyruvate is broken down to carbon dioxide and water. This process, which does require oxygen and is referred to as “oxidative respiration”, involves the stepwise metabolic breakdown of pyruvate by various enzymes of the Krebs tricarboxylic acid cycle and conversion of the products into high-energy molecules by electron transport chain reactions.

ATP, the energy source for the cell to function is ultimately formed when adenosine diphosphate (ADP), adds another phosphate group to form ATP. ATP cannot be stored in tissues in excess of a very limited threshold.

PARP-1

Multicellular organisms must have means of preserving their genomic integrity or face catastrophic consequences such as uncontrolled cell proliferation or massive cell death. One response is a modification of nuclear proteins by the addition and removal of polymers of ADP-ribose that modulate the properties of DNA-binding proteins involved in DNA repair and metabolism. These ADP-ribose units are added by poly(ADP-ribose) polymerase (PARP) and removed by poly(ADP-ribose) glycohydrolase(PARG).

Poly(ADP-ribose) polymerases (PARPs) are defined as cell signaling enzymes that catalyze the transfer of ADP-ribose units from NAD(+) to a number of acceptor proteins. PARP-1, the best characterized member of the PARP family, that presently includes six members, is an abundant nuclear enzyme implicated in cellular responses to DNA injury provoked by genotoxic stress (oxygen radicals, ionizing radiations and monofunctional alkylating agents). Due to its involvement either in DNA repair or in cell death, PARP-1 is regarded as a double-edged regulator of cellular functions. In fact, when the DNA damage is moderate, PARP-1 participates in the DNA repair process. Conversely, in the case of massive DNA injury, overactivation of PARP consumes NAD(+) and consequently ATP, culminating in cell dysfunction or necrosis. This cellular suicide mechanism has been implicated in the pathomechanism of stroke, myocardial ischemia, diabetes, diabetes-associated cardiovascular dysfunction, shock, traumatic central nervous system injury, arthritis, colitis, allergic encephalomyelitis, and various other forms of inflammation.

Living organisms possess mechanisms to regulate cell cycle progression and to preserve genomic integrity. Failure of these mechanisms in multicellular organisms results in disorders ranging from the unregulated cell proliferation associated with cancer to massive cell death after the fall of tissue oxygen and glucose levels in cardiac or brain ischemia. A key cellular response to genomic damage is the posttranslational modification of nuclear proteins in response to DNA strand breaks. One known modification is the addition to specific proteins of up to 200 residues of ADP-ribose to form branched polymers. These polymers act as binding sites for repair proteins that play a central role in DNA metabolism.

The enzyme responsible for the addition of these polymers is PARP1. PARP1 associates with DNA and with chromatin-binding proteins such as histones, transcription factors, and key DNA repair proteins. Although a number of nuclear proteins such as histones are substrates for PARP1, a major substrate is PARP1 itself, via automodification of the BRCA1 COOH-terminal homology region. Regulation of automodification of PARP1 is twofold: through PARP1-DNA interactions and PARP1-PARP1 dimerization. PARP1 acts together with the DNA damage repair system to regulate DNA base excision repair, apoptosis, and necrosis.

PARP-1 Inhibition

Studies of mouse strains lacking the PARP1 gene have identified two roles for this encoded protein, depending on the extent of DNA damage. Moderate damage elicits a protection response similar to that observed for checkpoint genes, leaving PARP1 knockout mice vulnerable to g-irradiation and alkylating reagents. In cases of extensive DNA damage, PARP1 activity depletes cellular energy pools, which eventually leads to cell death. PARP1 also has a putative role in signaling DNA damage and in recruiting proteins to sites of double-strand breaks. This hypothesis was based on the ability of proteins, such as p53 and other repair enzymes, to bind to the poly(ADP) polymers present on PARP1. PARP1 inhibitors exaggerate the cytotoxic effects of DNA damage by limiting the ability of cells to regulate DNA base excision repair. In this role, PARP inhibitors are being tested as chemosensitizing agents during cancer chemotherapy.

Another response to more extensive DNA damage mediated by PARP1 is the promotion of cell death, as seen in cases of ischemic injury. This process can occur when PARP1 activation is highly stimulated and thus consumes large amounts of NAD, the source of ADP-ribose. This condition depletes the cellular energy stores. PARP1 knockout mice are highly resistant to ischemia during stepto-zocin-induced type I diabetes, myocardial infarction, stroke, and neurodegeneration.

In support of a role for PARP1 in cell death in various inflammation processes, several studies have shown protection against cellular injury in numerous target cells by using known PARP1 inhibitors. For many years PARP1 has been the only known PARP. However, modification of cellular proteins with ADP-ribose polymers still occurs in PARP1 knockout mice, suggesting the presence of other proteins with PARP activity. Indeed, new members of the PARP family have been identified based on the presence of domains that share considerable sequence similarity with the catalytic domain of PARP1.

Although some members of the PARP family do not possess a well-defined Zn 21 finger DNA-binding motif or an auto-modification domain such as that described for PARP1, they still catalyze the formation of ADP-ribose polymers in a DNA-dependent manner and are capable of automodification. Two additional members of the PARP family are tankyrase and VPARP.

Tankyrase is associated with the telomerase complex that acts to regulate telomere length at replication, and VPARP is a component of a multisubunit complex referred to as a “vault”. The name vault is based on its observed structure by electron microscopy. The cellular location of VPARP is predominantly cytoplasmic; however, there is a small fraction associated with the mitotic spindle. Unlike PARP1, tankyrase and VPARP are not activated by DNA damage. Tankyrase modifies the telomere-binding protein TRF1 in vitro. TRF1 stabilizes the ends of chromosomes, and it has been proposed that modification of TRF1 with ADP-ribose polymers serves to regulate its ability to form a loop structure at chromosome ends. In other studies, tankyrase has been shown to promote telomere elongation in human cells. A substrate of VPARP is the major vault protein, MVP (it is also capable of automodification); these complexes are up-regulated in multidrug-resistant cancer cell lines. The various cellular locations and domain structures of the PARP family members strongly suggest that they have distinct cellular roles.

Identification of selective inhibitors might help elucidate the function of these enzymes. Poly(ADP-ribose) polymers can be removed by PARG, a member of a large family of related enzymes. This enzyme is thought to regulate the cellular function of PARP family members by removing ADP-ribose units, which results in changes in the branching pattern of the polymers. There is some evidence to support the hypothesis that polymers synthesized by different PARP orthologues might be hydrolyzed by specific PARGs. Although a complete understanding of the physiological activities of PARPs remains unclear, inhibitors of the activity of PARP1 and related proteins could provide new therapeutic approaches to both cancer and ischemia caused by reperfusion injury and inflammatory processes.

Excessive activation of poly(ADP-ribose) polymerase 1 (PARP1) leads to NAD(+) depletion and cell death during ischemia and other conditions that generate extensive DNA damage. When activated by DNA strand breaks, PARP1 uses NAD(+) as substrate to form ADP-ribose polymers on specific acceptor proteins. These polymers are in turn rapidly degraded by poly(ADP-ribose) glycohydrolase (PARG), a ubiquitously expressed exo- and endoglycohydrolase.

In a study, the role of PARG was examined in the PARP1-mediated cell death pathway. Mouse neuron and astrocyte cultures were exposed to hydrogen peroxide, N-methyl-d-aspartate (NMDA), or the DNA alkylating agent, N-methyl-N′-nitro-N-nitrosoguanidine (MNNG). Cell death in each condition was markedly reduced by the PARP1 inhibitor benzamide and equally reduced by the PARG inhibitors gallotannin and nobotanin B. The PARP1 inhibitor benzamide and the PARG inhibitor gallotannin both prevented the NAD(+) depletion that otherwise results from PARP1 activation by MNNG or H(2)O(2). However, these agents had opposite effects on protein poly(ADP-ribosyl)ation. Immunostaining for poly(ADP-ribose) on Western blots and neuron cultures showed benzamide to decrease and gallotannin to increase poly(ADP-ribose) accumulation during MNNG exposure.

These results suggest that PARG inhibitors do not inhibit PARP1 directly, but instead prevent PARP1-mediated celideath by slowing the turnover of poly(ADP-ribose) and thus slowing NAD(+) consumption. PARG appears to be a necessary component of the PARP-mediated cell death pathway, and PARG inhibitors may have promise as neuroprotective agents.

PARP-1 Support

“NAD+repletion prevents PARP-1-induced glycolytic blockade and cell death in cultured mouse astrocytes”, Biochem Biophys Res Commun. 2003 Sep. 5;308(4):809-13. Ying W, Garnier P, Swanson R A. Department of Neurology, University of California at San Francisco and Veterans Affairs Medical Center, 4150 Clement Street, San Francisco, Calif. 94121, USA.

Poly(ADP-ribose) polymerase-1 (PARP-1) is a nuclear enzyme that is involved in DNA repair and activated by DNA damage. When activated, PARP-1 consumes NAD(+) to form ADP-ribose polymers on acceptor proteins. Extensive activation of PARP-1 leads to glycolytic blockade, energy failure, and cell death. These events have been postulated to result from NAD(+) depletion. Here, we used primary astrocyte cultures to directly test this proposal, utilizing the endogenous expression of connexin-43 hemichannels by astrocytes to manipulate intracellular NAD(+) concentrations. Activation of PARP-1 with the DNA alkylating agent N-methyl-N′-nitro-N-nitrosoguanidine (MNNG) produced NAD(+) depletion, glycolytic blockade, and cell death. Cultures incubated in high (10 mM) extracellular concentrations of NAD(+) after MNNG exposure showed normalization of intracellular NAD(+) concentrations. Repletion of intracellular NAD(+) in this manner completely restored glycolytic capacity and prevented cell death. These results suggest that NAD(+) depletion is the cause of glycolytic failure after PARP-1 activation.

Extensive activation of poly(ADP-ribose) polymerase-1 (PARP-1) by DNA damage is a major cause of caspase-independent cell death in ischemia and inflammation. Here it is shown that NAD(+) depletion and mitochondrial permeability transition (MPT) are sequential and necessary steps in PARP-1-mediated cell death. Cultured mouse astrocytes were treated with the cytotoxic concentrations of N-methyl-N′-nitro-N-nitrosoguanidine or 3-morpholinosydnonimine to induce DNA damage and PARP-1 activation. The resulting cell death was preceded by NAD(+) depletion, mitochondrial membrane depolarization, and MPT. Sub-micromolar concentrations of cyclosporin A blocked MPT and cell death, suggesting that MPT is a necessary step linking PARP-1 activation to cell death. In astrocytes, extracellular NAD(+) can raise intracellular NAD(+) concentrations.

To determine whether NAD(+) depletion is necessary for PARP-1-induced MPT, NAD(+) was restored to near-normal levels after PARP-1 activation. Restoration of NAD(+) enabled the recovery of mitochondrial membrane potential and blocked both MPT and cell death. Furthermore, both cyclosporin A and NAD(+) blocked translocation of the apoptosis-inducing factor from mitochondria to nuclei, a step previously shown necessary for PARP-1-induced cell death. These results suggest that NAD(+) depletion and MPT are necessary intermediary steps linking PARP-1 activation to AIF translocation and cell death.

“Tricarboxylic acid cycle substrates prevent PARP-mediated death of neurons and astrocytes”, J Virol. 2004 September;78(18):9936-46., Ohsaki E, Ueda K, Sakakibara S, Do E, Yada K, Yamanishi K, Department of Microbiology, Osaka Graduate School of Medicine, 2-2 Yamada-oka, Suita, Osaka 565-0871, Japan.

The DNA repair enzyme, poly(ADP-ribose) polymerase-1 (PARP1), contributes to cell death during ischemia/reperfusion when extensively activated by DNA damage. The cell death resulting from PARP1 activation is linked to NAD+ depletion and energy failure, but [ . . . ] Because glycolysis requires cytosolic NAD+, the authors tested whether PARP1 activation impairs glycolytic flux and whether substrates that bypass glycolysis can rescue cells after PARP1 activation. PARP1 was activated in mouse cortical astrocyte and astrocyte-neuron cocultures [ . . . ] or other mitochondrial substrates to the cultures after MNNG treatment reduced cell death from approximately 70% to near basal levels, while PARP inhibitors and excess glucose had negligible effects. The mitochondrial substrates significantly reduced cell death.

PARP-1 and HIV

“Poly(ADP-ribose) polymerase-1 is a negative regulator of HIV-1 transcription through competitive binding to TAR RNA with Tat-P-TEFb complex.” J Biol Chem. 2004 Oct. 21, by Parent M, Yung T M, Rancourt A, Ho E L, Vispe S, Suzuki-Matsuda F, UeharaA, Wada T, Handa H, Satoh M S, from the Anatomy and Physiology, Faculty of Medicine, Laval University, Ste-Foy, QCG1 V 4G2.

Human immunodeficiency virus type-1 (HIV-1) transcription is regulated by a virus-encoded protein, Tat, which forms a complex with a host cellular factor, P-TEFb. When this complex binds to the TAR RNA synthesized from the HIV-1 long terminal repeat promoter element, transcription is trans-activated. Here, we show that, in host cells, HIV-1 transcription is negatively regulated by competition of poly(ADP-ribose) polymerase-1 (PARP-1) with Tat-P-TEFb for binding to TAR RNA. PARP-1, which has a high affinity for TAR RNA (KD=1.35×10−10 M), binds to the loop region of TAR RNA and displaces Tat or Tat-P-TEFb from the RNA. In vitro transcription assays have shown that this displacement leads to suppression of Tat-mediated trans-activation of transcription. Furthermore, in vivo expression of luciferase or destabilized enhanced green fluorescent protein genes under the control of the HIV-1 long terminal repeat promoter was suppressed by PARP-1. Thus, these results suggest that PARP-1 acts as a negative regulator of HIV-1 transcription through competitive binding with Tat or the Tat-P-TEFb complex to TAR RNA.

PPAR

Peroxisomal proliferator-activated receptors (PPARs) belong to a nuclear receptor superfamily of ligand-activated transcription factors. Peroxisome proliferator-activated receptor (PPAR) is activated when a ligand binds to the ligand-binding domain at the side of C-termini.

So far, three types of isoforms of alpha form, gamma form and delta form have been identified as PPARs, and the expression tissues and the functions are different respectively. Peroxisome proliferators are a structurally diverse group of compounds which, when administered to rodents, elicit dramatic increases in the size and number of hepatic and renal peroxisomes, as well as concomitant increases in the capacity of peroxisomes to metabolize fatty acids via increased expression of the enzymes required for the beta-oxidation cycle

It is known that the alpha-isoform of peroxisome proliferator-activated receptor (PPAR.alpha) acts to stimulate peroxisomal proliferation in the rodent liver which leads to enhanced fatty oxidation by this organ. (PPAR) alpha is a nuclear receptor that is mainly expressed in tissues with a high degree of fatty acid oxidation such as liver, heart, and skeletal muscle. There is a sex difference in PPARalpha expression. Male rats have higher levels of hepatic PPARalpha mRNA and protein than female rats. Chemicals included in this group are the fibrate class of hypolipidermic drugs, herbicides, and phthalate plasticizers.

Peroxisome proliferation can also be elicited by dietary or physiological factors such as a high-fat diet and cold acclimatization. The importance of peroxisomes in humans is stressed by the existence of a group of genetic diseases in man in which one or more peroxisomal functions are impaired. Most of the functions known to take place in peroxisomes have to do with lipids. Indeed, peroxisomes are capable of 1. fatty acid beta-oxidation 2. fatty acid alpha-oxidation 3. synthesis of cholesterol and other isoprenoids 4. ether-phospholipid synthesis and 5. biosynthesis of polyunsaturated fatty acids.

Peroxisome proliferator-activated receptors (PPAR) are nuclear receptors present in several organs and cell types. They are subdivided into PPAR alpha, PPAR gamma and PPAR delta (or beta). PPAR alpha and gamma are the two main categories of these receptors, which are both characterized by their ability to influence lipid metabolism, glucose homeostasis, cell proliferation, differentiation and apoptosis, as well as the inflammatory response, by transcriptional activation of target genes. PPAR alpha are activated by fatty acids, eicosanoids and fibrates, while PPAR gamma activators include arachidonic acid metabolites, oxidized low density lipoprotein and thiazolidinediones. PPAR gamma is predominantly expressed in intestine and adipose tissue, where it triggers adipocyte differentiation and promotes lipid storage. Recently, the expression of PPAR alpha and PPAR gamma was also reported in cells of the vascular wall, such as monocyte/macrophages, endothelial and smooth muscle cells.

The hypolipidemic fibrates and the antidiabetic glitazones are synthetic ligands for PPAR alpha and PPAR gamma, respectively. Furthermore, fatty acid-derivatives and eicosanoids are natural PPAR ligands: PPAR alpha is activated by leukotriene B4, whereas prostaglandin J2 is a PPAR gamma ligand, as well as some components of oxidized LDL, such as 9- and 1 3-HODE. These observations suggested a potential role for PPARs not only in metabolic but also in inflammation control and, by consequence, in related diseases such as atherosclerosis. More recently, PPAR activators were shown to inhibit the activation of inflammatory response genes (such as IL-2, IL-6, IL-8, TNF alpha and metalloproteases) by negatively interfering with the NF-kappa B, STAT and AP-1 signaling pathways in cells of the vascular wall.

The PPAR alpha form has been shown to mediate the action of the hypolipidemic drugs of the fibrate class on lipid and lipoprotein metabolism. PPAR alpha activators furthermore improve glucose homeostasis and influence body weight and energy homeostasis. It is likely that these actions of PPAR alpha activators on lipid, glucose and energy metabolism are, at least in part, due to the increase of hepatic fatty acid beta-oxidation resulting in an enhanced fatty acid flux and degradation in the liver.

Moreover, PPARs are expressed in different immunological and vascular wall cell types where they exert anti-inflammatory and proapoptotic activities. The observation that these receptors are also expressed in atherosclerotic lesions suggests a role in atherogenesis. Finally, PPAR alpha activators correct age-related dysregulations in redox balance. Taken together, these data indicate a modulatory role for PPAR alpha in the pathogenesis of age-related disorders, such as dyslipidemia, insulin resistance and chronic inflammation, predisposing to atherosclerosis.

Synthetic antidiabetic thiazolidinediones (TZDs) (two such compounds are rosiglitazone and pioglitazone) and natural prostaglandin D(2) (PGD(2)) metabolite, 15-deoxy-Delta(12, 14)-prostaglandin J(2) (15d-PGJ(2)), are well-known as ligands for PPAR gamma. After it has been reported that activation of PPAR gamma suppresses production of proinflammatory cytokines in activated macrophages, medical interest in PPAR gamma have grown and a huge research effort has been concentrated. PPAR gamma, is currently known to be implicated in various human chronic diseases such as diabetes mellitus, atherosclerosis, rheumatoid arthritis, inflammatory bowel disease, and Alzheimer's disease.

Moreover, PPAR gamma ligands have potent tumor modulatory effects against colorectal, prostate, and breast cancers. Recent studies suggest that TZDs not only ameliorate insulin sensitivity but also have pleiotropic effects on many tissues and cell types. Although activation of PPAR gamma seems to have beneficial effects on atherosclerosis and heart failure, the mechanisms by which PPAR gamma ligands prevent the development of cardiovascular diseases are not fully understood.

The PPAR gamma agonist ciglitazone inhibited HIV-1 replication in a dose-dependent manner in acutely infected human MDM by transcriptional and post-transcriptional effects. Ciglitazone also suppressed HIV-1 mRNA levels as measured by reverse transcriptase PCR, in parallel with the decrease in reverse transcriptase activity. Co-transfection of PPAR gamma wild type vectors and treatment with PPAR gamma agonists inhibited HIV-1 promoter activity in U937 cells. HIV nuclear import, DNA integration, chromatin template capacity may be mediated by the lipid environment. PPAR agonists effect on the lipid-enriched (HIV-1 infection induces alteration of cellular lipids) microdomains from which HIV-1 buds, (may explain the high level of cholesterol and sphingolipids in the viral envelope, since host cell rafts become a viral coat) offers interesting future therapy.

Monocytes/macrophages (Mphi) play a pivotal role in the persistence of chronic inflammation and local tissue destruction in diseases such as rheumatoid arthritis and atherosclerosis. The production by Mphi of cytokines, chemokines, metalloproteinases and their inhibitors is an essential component in this process, which is tightly regulated by multiple factors. The peroxisome proliferator-activated receptors (PPARs) were shown to be involved in modulating inflammation.

PPAR gamma is activated by a wide variety of ligands such as fatty acids, the anti-diabetic thiazolidinediones (TZDs), and also by certain prostaglandins of which 15-deoxy-Delta(12, 14)-PGJ2 (PGJ2). High concentrations of PPAR gamma ligands were shown to have anti-inflammatory activities by inhibiting the secretion of interleukin-1 (IL-1), interleukin-6 (IL-6) and tumour necrosis factor alpha (TNFalpha) by stimulated monocytes.

The aim of this study was to determine whether PGJ2 and TZDs would also exert an immunomodulatory action through the up-regulation of anti-inflammatory cytokines such as the IL-1 receptor antagonist (IL-1Ra). THP-1 monocytic cells were stimulated with PMA, thereby enhancing the secretion of IL-1, IL-6, TNFalpha, IL-1Ra and metalloproteinases. Addition of PGJ2 had an inhibitory effect on IL-1, IL-6 and TNFalpha secretion, while increasing IL-1Ra production. In contrast, the bona fide PPAR gamma ligands (TZDs; rosiglitazone, pioglitazone and troglitazone) barely inhibited proinflammatory cytokines, but strongly enhanced the production of IL-1Ra from PMA-stimulated THP-1 cells. Unstimulated cells did not respond to TZDs in terms of IL-1Ra production, suggesting that in order to be effective, PPAR ligands depend on PMA signalling. Basal levels of PPAR gamma are barely detectable in unstimulated THP-1 cells, while stimulation with PMA up-regulates its expression, suggesting that higher levels of PPAR gamma expression are necessary for receptor ligand effects to occur.

In conclusion, it was demonstrated for the first time that TZDs may exert an anti-inflammatory activity by inducing the production of the IL-1Ra.

Peroxisome proliferator-activated receptors (PPARs) are ligand-activated transcription factors that directly control numerous genes of lipid metabolism by binding to response elements in the promoter. It has recently been proposed that PPARgamma may also regulate genes for proinflammatory proteins, not through PPRE binding but by interaction with transcription factors AP-1, STAT, and NF-kappaB.

Recent studies with cultured human monocytes, however, have failed to observe an inhibitory effect of PPARgamma agonists on induced expression of TNFalpha and IL-6, genes known to be controlled by AP-1, STAT, and NF-kappaB. In a similar fashion, we show here that PPARalpha (fenofibrate) or PPARgamma (rosiglitazone) agonists failed to modulate LPS-induced secretion of IL-8 in THP-1 cells. When we made parallel observations on another gene, matrix metalloproteinase 9 (MMP-9), we were surprised to find profound downregulation of LPS-induced secretion by both PPARalpha or PPARgamma agonists. These findings suggest that PPAR may regulate only a subset of the proinflammatory genes controlled by AP-1, STAT, and NF-kappaB. Effects of PPARs on MMP-9 may account for the beneficial effect of PPAR agonists in animal models of atherosclerosis.

Metabolic effects of rosiglitazone in HIV lipodystrophy: a randomized, controlled trial”. “Hadigan C, Yawetz S, Thomas A, Havers F, Sax P E, Grinspoon S. Massachusetts General Hospital and Brigham and Women's Hospital, Boston, Mass. 02114, USA.

BACKGROUND: Patients with HIV infection who are treated with antiretroviral agents often lose subcutaneous fat and have metabolic abnormalities, including insulin resistance and reduced adiponectin levels, which may be related to disrupted subcutaneous adipogenesis and altered peroxisome proliferator-activated receptor-gamma signaling.

OBJECTIVE: To investigate the effects of rosiglitazone (4 mg/d), a peroxisome proliferator-activated receptor-gamma agonist, in HIV-infected men and women with hyperinsulinemia and lipoatrophy.

DESIGN: A randomized, double-blind, placebo-controlled, 3-month study. SETTING: University hospital.

PATIENTS: 28 HIV-infected men and women with hyperinsulinemia and lipoatrophy.

MEASUREMENTS: Insulin sensitivity measured by euglycemic hyperinsulinemic clamp testing; subcutaneous leg fat area measured by computed tomography; adiponectin, free fatty acid, and lipid levels; and safety variables.

RESULTS: Rosiglitazone, when compared with placebo, improved insulin sensitivity (mean [+/−SD] change, 1.5+/−2.1 mg of glucose/kg of lean body mass per minute vs. −0.4+/−1.6 mg/kg per minute; P=0.02), increased adiponectin levels (mean [+/−SD], 2.2+/−2.2 micro g/mL vs. 0.1+/−1.1 microg/mL; P=0.006), and reduced free fatty acid levels (mean [+/−SD], −0.09+/−0.1 mmol/L vs. 0.01+/−0.1 mmol/L; P=0.02). Mean percentage (+/−SD) of body fat (1.38%+/−3.03% vs. −0.83%+/−2.76%; P=0.03) and subcutaneous leg fat area (2.3+/−8.4 cm2 vs. −0.9+/−1.9 cm2; P=0.02) increased significantly with rosiglitazone compared with placebo. Mean total cholesterol levels (+/−SD) also increased with rosiglitazone compared with placebo (0.6+/−1.0 mmol/L [25+/−37 mg/dL] vs. −0.4+/−0.6 mmol/L [−15+/−25 mg/dL]; P=0.007). LIMITATIONS: The study was relatively small and of short duration.

CONCLUSIONS: The authors demonstrated positive effects of rosiglitazone on lipoatrophy; insulin sensitivity; and metabolic indices, including adiponectin levels, in HIV-infected patients with lipoatrophy and insulin resistance. Peroxisome proliferator-activated receptor-gamma agonists may correct the metabolic abnormalities associated with disrupted adipogenesis in this population. Further studies must determine the clinical utility of such agents in HIV-infected patients.

Diabetes. 2004 August;53(8):2169-76.” Effects of rosiglitazone and metformin on liver fat content, hepatic insulin resistance, insulin clearance, and gene expression in adipose tissue in patients with type 2 diabetes.” Tiikkainen M, Hakkinen A M, Korsheninnikova E, Nyman T, Makimattila S, Yki-Jarvinen H. Department of Medicine, University of Helsinki, Helsinki, Finland.

Both rosiglitazone and metformin increase hepatic insulin sensitivity, but their mechanism of action has not been compared in humans. The objective of this study was to compare the effects of rosiglitazone and metformin treatment on liver fat content, hepatic insulin sensitivity, insulin clearance, and gene expression in adipose tissue and serum adiponectin concentrations in type 2 diabetes.

A total of 20 drug-naive patients with type 2 diabetes (age 48+/−3 years, fasting plasma glucose 152+/−9 mg/dl, BMI 30.6+/−0.8 kg/m2) were treated in a double-blind randomized fashion with either 8 mg rosiglitazone or 2 g metformin for 16 weeks. Both drugs similarly decreased HbA1c, insulin, and free fatty acid concentrations. Body weight decreased in the metformin (84+/−4 vs. 82+/−4 kg, P<0.05) but not the rosiglitazone group. Liver fat (proton spectroscopy) was decreased with rosiglitazone by 51% (15+/−3 vs. 7+/−1%, 0 vs. 16 weeks, P=0.003) but not by metformin (13+/−3 to 14+/−3%, NS). Rosiglitazone (16+/−2 vs. 20+/−1 ml.kg(−1).min(−1), P=0.02) but not metformin increased insulin clearance by 20%. Hepatic insulin sensitivity in the basal state increased similarly in both groups. Insulin-stimulated glucose uptake increased significantly with rosiglitazone but not with metformin. Serum adiponectin concentrations increased by 123% with rosiglitazone but remained unchanged during metformin treatment. The decrease of serum adiponectin concentrations correlated with the decrease in liver fat (r=−0.74, P<0.001). Rosiglitazone but not metformin significantly increased expression of peroxisome proliferator-activated receptor-gamma, adiponectin, and lipoprotein lipase in adipose tissue.

In conclusion, rosiglitazone but not metformin decreases liver fat and increases insulin clearance. The decrease in liver fat by rosiglitazone is associated with an increase in serum adiponectin concentrations. Both agents increase hepatic insulin sensitivity, but only rosiglitazone increases peripheral glucose uptake.

Arterioscler Thromb Vasc Biol. 2004 May;24(5):930-4. Epub 2004 Mar. 04. “Effect of rosiglitazone on common carotid intima-media thickness progression in coronary artery disease patients without diabetes mellitus.” Sidhu J S, Kaposzta Z, Markus HS, Kaski J C.Coronary Artery Disease Research Unit, St. George's Hospital Medical School, London, UK.

OBJECTIVE: Thiazolidinediones, such as rosiglitazone, have been shown to retard atherosclerosis disease progression in diabetic subjects. These agents may have anti-atherosclerotic effects through direct inhibition of inflammatory processes in the vessel wall, and so their benefit may extend to patients with atherosclerotic disease, even in the absence of diabetes. In this study, we assessed the effect of rosiglitazone on common carotid intima-media thickness (IMT) progression in nondiabetic coronary artery disease (CAD) patients.

METHODS AND RESULTS: Consecutive subjects (n=92) with clinically stable, angiographically documented CAD and without diabetes mellitus were randomized in a double-blind manner to receive placebo or rosiglitazone for 48 weeks. They received single-dose placebo and rosiglitazone 4 mg daily for the initial 8 weeks, and the doses were doubled for the remainder of the study. Common carotid IMT together with fasting glucose, insulin, and lipid profile were measured at baseline and repeated after 24 and 48 weeks. Rosiglitazone-treated patients showed reduced IMT progression compared with the placebo group, −0.012 mm/48 weeks versus 0.031 mm/48 weeks (P=0.03). Rosiglitazone treatment significantly reduced insulin resistance, estimated by homeostasis model of insulin resistance index, compared with placebo (P=0.01).

CONCLUSIONS: Rosiglitazone reduces common carotid IMT progression in nondiabetic CAD patients, and insulin-sensitization may be one contributory mechanism.

Am J Physiol Endocrinol Metab. 2004June;286(6):E941-9. Epub 2004 Jan. 28. “Effects of rosiglitazone on gene expression in subcutaneous adipose tissue in highly active antiretroviral therapy-associated lipodystrophy.” Sutinen J, Kannisto K, Korsheninnikova E, Fisher R M, Ehrenborg E, Nyman T, Virkamaki A, Funahashi T, Matsuzawa Y, Vidal H, Hamsten A, Yki-Jarvinen H. Division of Diabetes, Department of Medicine, Helsinki University Central Hospital, PO Box 348, FIN-00029 HUS, Helsinki, Finland.

Highly active antiretroviral therapy (HAART) has improved the prognosis of human immunodeficiency virus (HIV)-infected patients but is associated with severe adverse events, such as lipodystrophy and insulin resistance. Rosiglitazone did not increase subcutaneous fat in patients with HAART-associated lipodystrophy (HAL) in a randomized, double-blind, placebo-controlled trial, although it attenuated insulin resistance and decreased liver fat content.

The aim of this study was to examine effects of rosiglitazone on gene expression in subcutaneous adipose tissue in 30 patients with HAL. The mRNA concentrations in subcutaneous adipose tissue were measured using real-time PCR. Twenty-four-week treatment with rosiglitazone (8 mg/day) compared with placebo significantly increased the expression of adiponectin, peroxisome proliferator-activated receptor-gamma (PPARgamma), and PPARgamma coactivator 1 and decreased IL-6 expression. Expression of other genes involved in lipogenesis, fatty acid metabolism, or glucose transport, such as acyl-CoA synthase, adipocyte lipid-binding protein, CD45, fatty acid transport protein-1 and -4, GLUT1, GLUT4, keratinocyte lipid-binding protein, lipoprotein lipase, PPARdelta, and sterol regulatory element-binding protein-1c, remained unchanged. Rosiglitazone also significantly increased serum adiponectin concentration. The change in serum adiponectin concentration was inversely correlated with the change in fasting serum insulin concentration and liver fat content. In conclusion, rosiglitazone induced significant changes in gene expression in subcutaneous adipose tissue and ameliorated insulin resistance in patients with HAL. Increased expression of adiponectin might have mediated most of the favorable insulin-sensitizing effects of rosiglitazone in these patients.

J Biol Chem. 2004 Aug. 6;279(32):33456-62. Epub 2004 Jun. 03. “Isohumulones, bitter acids derived from hops, activate both peroxisome proliferator-activated receptor alpha and gamma and reduce insulin resistance”. Yajima H, Ikeshima E, Shiraki M, Kanaya T, Fujiwara D, Odai H, Tsuboyama-Kasaoka N, Ezaki O, Oikawa S, Kondo K. Central Laboratories for Key Technology, Kirin Brewery Co., Ltd., Kanagawa 236-0004, Japan.

The peroxisome proliferator-activated receptors (PPARs) are dietary lipid sensors that regulate fatty acid and carbohydrate metabolism. The hypolipidemic effects of fibrate drugs and the therapeutic benefits of the thiazolidinedione drugs are due to their activation of PPARalpha and -gamma, respectively. In this study, isohumulones, the bitter compounds derived from hops that are present in beer, were found to activate PPARalpha and -gamma in transient co-transfection studies. Among the three major isohumulone homologs, isohumulone and isocohumulone were found to activate PPARalpha and -gamma. Diabetic KK-Ay mice that were treated with isohumulones (isohumulone and isocohumulone) showed reduced plasma glucose, triglyceride, and free fatty acid levels (65.3, 62.6, and 73.1%, respectively, for isohumulone); similar reductions were found following treatment with the thiazolidinedione drug, pioglitazone. Isohumulone treatment did not result in significant body weight gain, although pioglitazone treatment did increase body weight (10.6% increase versus control group). C57BL/6N mice fed a high fat diet that were treated with isohumulones showed improved glucose tolerance and reduced insulin resistance.

Furthermore, these animals showed increased liver fatty acid oxidation and a decrease in size and an increase in apoptosis of their hypertrophic adipocytes. A double-blind, placebo-controlled pilot study for studying the effect of isohumulones on diabetes suggested that isohumulones significantly decreased blood glucose and hemoglobin A1c levels after 8 weeks (by 10.1 and 6.4%, respectively, versus week 0). These results suggest that isohumulones can improve insulin sensitivity in high fat diet-fed mice with insulin resistance and in patients with type 2 diabetes.

Diabetes Care. 2002 February;25(2):376-80. “Synthetic peroxisome proliferator-activated receptor-gamma agonist, rosiglitazone, increases plasma levels of adiponectin in type 2 diabetic patients”. Yang W S, Jeng C Y, Wu T J, Tanaka S, Funahashi T, Matsuzawa Y, Wang J P, Chen C L, Tai T Y, Chuang L M. Department of Internal Medicine, National Taiwan University Hospital, 7 Chung-Shan South Road, Taipei, Taiwan.

OBJECTIVE: Adiponectin, a plasma protein exclusively synthesized and secreted by adipose tissue, has recently been shown to have anti-inflammatory, antiatherogenic properties in vitro and beneficial metabolic effects in animals. Lower plasma levels of adiponectin have been documented in human subjects with metabolic syndrome and coronary artery disease. We investigated whether the level of this putative protective adipocytokine could be increased by treatment with a peroxisome proliferator-activated receptor-gamma (PPAR-gamma) agonist in diabetic patients.

RESEARCH DESIGN AND METHODS: Type 2 diabetic patients (30 in the treatment group and 34 in the placebo group) were recruited for a randomized double-blind placebo-controlled trial for 6 months with the PPAR-gamma agonist rosiglitazone. Blood samples were collected and metabolic variables and adiponectin levels were determined in all patients before initiation of the study.

RESULTS: In the rosiglitazone group, mean plasma adiponectin level was increased by more than twofold (P<0.0005), whereas no change was observed in the placebo group. Multivariate linear regression analysis showed that whether rosiglitazone was used was the single variable significantly related to the changes of plasma adiponectin. The amount of variance in changes of plasma adiponectin level explained by the treatment was approximately 24% (r(2)=0.24) after adjusting for age, sex, and changes in fasting plasma glucose, HbA(1c), insulin resistance index, and BMI. CONCLUSIONS: Rosiglitazone increases plasma adiponectin levels in type 2 diabetic subjects. Whether this may contribute to the antihyperglycemic and putative antiatherogenic benefits of PPAR-gamma agonists in type 2 diabetic patients warrants further investigation.

J Acquir Immune Defic Syndr. 2002 Oct. 1;31(2):163-70, “Improved insulin sensitivity and body fat distribution in HIV-infected patients treated with rosiglitazone: a pilot study”. Gelato M C, Mynarcik D C, Quick J L, Steigbigel R T, Fuhrer J, Brathwaite C E, Brebbia J S, Wax M R, McNurlan M A. Department of Medicine, State University of New York at Stony Brook, 11794-8154, USA.

The insulin-sensitizing drugs thiazolidinediones (TZDs), such as rosiglitazone, improve insulin sensitivity and also promote adipocyte differentiation in vitro. The authors hypothesized that TZDs might be beneficial to patients with HIV disease to improve insulin sensitivity and the distribution of body fat by increasing peripheral fat. The ability of rosiglitazone (8 mg/d) to improve insulin sensitivity (from hyperinsulinemic-euglycemic clamp) and to improve body fat distribution (determined from computed tomography measurements of visceral adipose tissue [VAT] and subcutaneous adipose tissue [SAT]) was determined in 8 HIV-positive patients.

Before treatment, the insulin sensitivity of the patients was reduced to approximately 34% of that in control subjects. The rate of glucose disposal during a hyperinsulinemic-euglycemic clamp (Rd) was 3.8+/−0.4 (SEM) mg glucose/kg lean body mass/min compared with 11.08+/−1.1 (p<0.001) in healthy age- and body mass index (BMI)-matched control subjects. After rosiglitazone treatment of 6 to 1 2 weeks, Rd increased to 5.99+/−0.9 (p=0.02), an improvement of 59+/−22%. SAT increased by 23+/−10% (p=0.05), and, surprisingly, VAT was decreased by 21+/−8% (p=0.04) with a trend for increased SAT/VAT that failed to reach statistical significance. There were no significant changes in blood counts, viral loads, or CD4 counts with rosiglitazone treatment. The study demonstrates that rosiglitazone therapy improves insulin resistance and body fat distribution in some patients with HIV disease.

Am J Hypertens. 2003 October; 16(10):894. “Treatment with rosiglitazone reduces hyperinsulinemia and improves arterial elasticity in patients with type 2 diabetes mellitus”. Shargorodsky M, Wainstein J, Gavish D, Leibovitz E, Matas Z, Zimlichman R, Wainstein G, Gavish E, Leibovitz Z, Matas D. Department of Endocrinology and Diabetes, Wolfson Medical Center, Holon 581 00, Israel.

OBJECTIVE: The aim of this study was to determine whether reduction of hyperinsulinemia with rosiglitazone will improve vascular elasticity in patients with non-insulin dependent diabetes mellitus.

METHODS: In an open label study 52 patients with non-insulin dependent diabetes mellitus and at least one additional cardiovascular risk factor, were treated for 6 months with 4 mg of rosiglitazone, and uptitrated to 8 mg after 3 months of treatment, if needed. At the beginning of the study and at its end, blood was drawn for insulin, C-peptide, and 24-h urine collected for microalbuminuria/proteinuria. Glucose, chemistry, lipid profile, and hemoglobin A1C were determined at 0, 3, and 6 months. Vascular compliance was measured in monthly intervals.

RESULTS: Treatment increased significantly small artery elasticity from 1.45 to 2.43 mL/mm Hg×100. Large artery elasticity tended to increase toward the end of the study (P=not significant). Systolic blood pressure (BP) decreased from 144 to 124 mm Hg and diastolic BP decreased from 80 to 62.5 mm Hg, despite mild weight gain [corrected]. Heart rate tended to decrease from 76.3 to 74.7 beats/min (P=not significant). Systemic vascular resistance decreased from 1789.8 to 1329.4 dyne sec/cm(5). Plasma insulin, in patients not treated with insulin, decreased from 42.45+/−24.90 to 27.86+/−14.86 IU/mL (P=0.0001).

CONCLUSIONS: Treatment with rosiglitazone reduced hyperinsulinemia and improved small artery elasticity with a tendency to improve large artery elasticity, in hypertensive and in normotensive patients. Because rosiglitazone improves insulin receptor sensitivity (IRS), it is logical to assume that the reduction in hyperinsulinemia reflects improvement in IRS. Our data support the hypothesis that hyperinsulinemia and IRS participate in the mechanisms of tissue injury and their improvement induces improvement in arterial elasticity.

Br J Pharmacol. 2004 Nov. 8 [Epub ahead of print] “GW9662, a potent antagonist of PPAR{gamma}, inhibits growth of breast tumour cells and promotes the anticancer effects of the PPAR{gamma} agonist rosiglitazone, independently of PPAR{gamma} activation.” Seargent J M, Yates E A, Gill J H.

Peroxisome proliferator-activated receptor gamma (PPARgamma), a member of the nuclear receptor superfamily, is activated by several compounds, including the thiazolidinediones. In addition to being a therapeutic target for obesity, hypolipidaemia and diabetes, perturbation of PPARgamma signalling is now believed to be a strategy for treatment of several cancers, including breast.

Although differential expression of PPARgamma is observed in tumours compared to normal tissues and PPARgamma agonists have been shown to inhibit tumour cell growth and survival, the interdependence of these observations is unclear. This study demonstrated that the potent, irreversible and selective PPARgamma antagonist GW9662 prevented activation of PPARgamma and inhibited growth of human mammary tumour cell lines. Controversially, GW9662 prevented rosiglitazone-mediated PPARgamma activation, but enhanced rather than reversed rosiglitazone-induced growth inhibition. As such, these data support the existence of PPARgamma-independent pathways and question the central belief that PPARgamma ligands mediate their anticancer effects via activation of PPARgamma.

Genes Cells. 2004 November;9(11):1113-23. “Peroxisome proliferator-activated receptor gamma-dependent and -independent growth inhibition of gastrointestinal tumour cells.” Rumi M A, Ishihara S, Kadowaki Y, Ortega-Cava C F, Kazumori H, Kawashima K, Yoshino N, Yuki T, Ishimura N, Kinoshita Y. Department of Gastroenterology and Hepatology, Shimane University School of Medicine, Izumo-City, Shimane 693-8501, Japan.

Peroxisome proliferator-activated receptor gamma (PPARgamma) acts as a ligand-activated transcription factor. Although ligand-induced cellular differentiation and growth inhibition have been mostly studied on human cancers expressing PPARgamma, it is unclear if the transcriptional activation of PPARgamma is the main mechanism of growth inhibition. In this study, we investigated whether there is a link between growth inhibitory effect and transcriptional activation of PPARgamma in several gastrointestinal tumour cell lines. The transcriptional activation potential of PPARgamma was assessed by reporter gene assay employing a PPRE-luciferase vector, and growth inhibitory effect of PPARgamma was investigated by (3) H-thymidine incorporation assay, in the presence or absence of thiazolidinedione ligands, rosiglitazone and troglitazone.

As expected, in the case of cell lines positive for the transcriptional activation potential of PPARgamma (T.Tn, MKN-45 and LoVo), both the ligands induced growth inhibition. However, in case of some other cell lines negative for the transcriptional activation potential of PPARgamma (TT, AGS and HCT-15), troglitazone still showed a growth inhibitory effect. Administration of the PPARgamma antagonist GW9662 did not reverse this growth inhibitory activity of troglitazone. The introduction of dominant negative mutants of PPARgamma did not suppress the activity either. These observations suggest that while rosiglitazone inhibits cellular growth predominantly through transcriptional activation of PPARgamma, troglitazone can induce it both in PPARgamma-dependent and -independent pathways.

Int J Oncol. 2004 August;25(2):493-502. “The PPARgamma ligands PGJ2 and rosiglitazone show a differential ability to inhibit proliferation and to induce apoptosis and differentiation of human glioblastoma cell lines”. Morosetti R, Servidei T, Mirabella M, Rutella S, Mangiola A, Maira G, Mastrangelo R, Koeffler H P. Division of Pediatric Oncology, Catholic University of Rome, 00168 Rome, Italy.

Peroxisome proliferator-activated receptor gamma (PPARgamma) is involved in the control of cell proliferation, apoptosis and differentiation in various tumor cells. Among PPARgamma ligands, 15-deoxy-Delta12,14-prostaglandin J2 (PGJ2), the ultimate metabolite of PGD2, plays a role in the biology of brain tumors. It is still unclear to which extent the anti-proliferative and differentiation-promoting activity of PGJ2 is mediated through PPARgamma.

We compared the effects of PGJ2 with those of rosiglitazone—the synthetic agonist with the highest affinity for PPARgamma—in 4 human glioblastoma cell lines (A172, U87-MG, M059K, M059J). All cell lines expressed high levels of PPARgamma, consistent with the high levels of PPARgamma protein in 5 tumor samples. Both PGJ2 and rosiglitazone inhibited proliferation of all cell lines with a G2/M arrest and apoptosis, but only PGJ2 up-regulated p21 Cip/WAF1. The growth inhibitory effect was partially reversed by the PPARgamma antagonist GW9662.

We studied the time sequence of selected molecular events that lead glioblastoma cells to apoptosis and/or differentiation, after treatment with both agonists. M059K cells committed to undergo apoptosis by PGJ2, initially up-regulated PPARgamma, and then down-regulated PPARgamma as they began apoptosis. Apoptotic cells also increased their expression of retinoic acid receptor beta (RARbeta) and retinoid X receptor alpha (RXRalpha). PGJ2 increased expression of glial fibrillary acidic protein (GFAP) and decreased levels of vimentin, structural proteins modulated during astrocytic differentiation. Unexpectedly, PGJ2 up-regulated the expression of cyclooxygenase-2 (COX-2). Rosiglitazone caused the same pattern of PPARgamma, RARbeta and RXRalpha expression as PGJ2, but no significant modulation of p21Cip/WAF1, cytoskeletal proteins or COX-2 occurred. Our data indicate that PGJ2, and rosiglitazone suppress cell proliferation and cause apoptosis in glioblastoma cell lines, most likely through a PPARgamma-dependent pathway. By contrast, the modulation of differentiation-associated proteins by PGJ2, but not rosiglitazone, suggests that PGJ2 promotes differentiation of glioblastoma cells independently of PPARgamma activation.

Circ Res. 2003 Aug. 22;93(4):e38-47. Epub 2003 Jul. 24. “Regulation of the growth arrest and DNA damage-inducible gene 45 (GADD45) by peroxisome proliferator-activated receptor gamma in vascular smooth muscle cells.” Bruemmer D, Yin F, Liu J, Berger J P, Sakai T, Blaschke F, Fleck E, Van Herle A J, Forman B M, Law R E. Division of Endocrinology, Diabetes and Hypertension and The Gonda (Goldschmied) Diabetes Center, David Geffen School of Medicine, University of California, Los Angeles, Calif. 90095, USA.

Peroxisome proliferator-activated receptor (PPAR) gamma is activated by thiazolidinediones (TZDs), widely used as insulin-sensitizing agents for the treatment of type 2 diabetes. TZDs have been shown to induce apoptosis in a variety of mammalian cells. In vascular smooth muscle cells (VSMCs), proliferation and apoptosis may be competing processes during the formation of restenotic and atherosclerotic lesions. The precise molecular mechanisms by which TZDs induce apoptosis in VSMCs, however, remain unclear.

In the present study, we demonstrate that the TZDs rosiglitazone (RSG), troglitazone (TRO), and a novel non-TZD partial PPARgamma agonist (nTZDpa) induce caspase-mediated apoptosis of human coronary VSMCs. Induction of VSMC apoptosis correlated closely with an upregulation of growth arrest and DNA damage-inducible gene 45 (GADD45) mRNA expression and transcription, a well-recognized modulator of cell cycle arrest and apoptosis. Using adenoviral-mediated overexpression of a constitutively active PPARgamma mutant and the irreversible PPARgamma antagonist GW9662, we provide evidence that PPARgamma ligands induce caspase-mediated apoptosis and GADD45 expression through a receptor-dependent pathway. Deletion analysis of the GADD45 promoter revealed that a 153-bp region between −234 and −81 bp proximal to the transcription start site, containing an Oct-1 element, was crucial for the PPARgamma ligand-mediated induction of the GADD45 promoter. PPARgamma activation induced Oct-1 protein expression and DNA binding and stimulated activity of a reporter plasmid driven by multiple Oct-1 elements.

These findings suggest that activation of PARgamma can lead to apoptosis and growth arrest in VSMCs, at least in part, by inducing Oct-1-mediated transcription of GADD45.

Biochem Biophys Res Commun. 2004 Feb. 20;314(4):1093-9. “Suppression of prostaglandin E2 receptor subtype EP2 by PPARgamma ligands inhibits human lung carcinoma cell growth.” Han S, Roman J. Division of Pulmonary, Allergy and Critical Care Medicine, Department of Medicine, Emory University School of Medicine, Atlanta, Ga. 30322, USA.

Prostaglandin E(2) (PGE(2)), a major cyclooxygenase (COX-2) metabolite, plays important roles in tumor biology and its functions are mediated through one or more of its receptors EP1, EP2, EP3, and EP4. We have shown that the matrix glycoprotein fibronectin stimulates lung carcinoma cell proliferation via induction of COX-2 expression with subsequent PGE(2) protein biosynthesis. Ligands of peroxisome proliferator-activated receptor gamma (PPARgamma) inhibited this effect and induced cellular apoptosis. Here, we explore the role of the PGE(2) receptor EP2 in this process and whether the inhibition observed with PPARgamma ligands is related to effects on this receptor.

We found that human non-small cell lung carcinoma cell lines (H1838 and H2106) express EP2 receptors, and that the inhibition of cell growth by PPARgamma ligands (GW1929, PGJ2, ciglitazone, troglitazone, and rosiglitazone [also known as BRL49653]) was associated with a significant decrease in EP2 mRNA and protein levels. The inhibitory effects of BRL49653 and ciglitazone, but not PGJ2, were reversed by a specific PPARgamma antagonist GW9662, suggesting the involvement of PPARgamma-dependent and -independent mechanisms. PPARgamma ligand treatment was associated with phosphorylation of extracellular regulated kinase (Erk), and inhibition of EP2 receptor expression by PPARgamma ligands was prevented by PD98095, an inhibitor of the MEK-1/Erk pathway. Butaprost, an EP2 agonist, like exogenous PGE(2) dmPGE(2)), increased lung carcinoma cell growth, however, GW1929 and troglitazone blocked their effects. Our studies reveal a novel role for EP2 in mediating the proliferative effects of PGE(2) on lung carcinoma cells. PPARgamma ligands inhibit human lung carcinoma cell growth by decreasing the expression of EP2 receptors through Erk signaling and PPARgamma-dependent and -independent pathways.

J Pharmacol Exp Ther. 2003 November;307(2):505-17. Epub 2003 Sep. 09. Peroxisome proliferator-activated receptor-gamma activator 15-deoxy-Delta12,14-prostaglandin J2 inhibits neuroblastoma cell growth through induction of apoptosis: association with extracellular signal-regulated kinase signal pathway. Kim E J, Park K S, Chung S Y, Sheen Y Y, Moon D C, Song Y S, Kim K S, Song S, Yun Y P, Lee M K, Oh K W, Yoon do Y, Hong J T. National Institute of Toxicological Research, Korea Food and Drug Administration, Seoul, Korea.

Peroxisome proliferator-activated receptor-gamma (PPAR-gamma) ligands have been demonstrated to inhibit growth of several cancer cells. Here, we investigated whether one of the PPAR-gamma ligands, 15-deoxy-Delta12,14-prostaglandin J2 (15-deoxy-PGJ2) inhibits cell growth of two human neuroblastoma cells (SK-N-SH and SK-N-MC) in a PPAR-gamma-dependent manner. PPAR-gamma was expressed in these cells, and 15-deoxy-PGJ2 increased expression, DNA binding activity, and transcriptional activity of PPAR-gamma. 15-Deoxy-PGJ2 also inhibited cell growth in time- and dose-dependent manners in both cells. Cells were arrested in G2/M phase after 15-deoxy-PGJ2 treatment with concomitant increase in the expression of G2/M phase regulatory protein cyclin B1 but decrease in the expression of cdk2, cdk4, cyclin A, cyclin D1, cyclin E, and cdc25C.

Conversely, related to the growth inhibitory effect, 15-deoxy-PGJ2 increased the induction of apoptosis in a dose-dependent manner. Consistent with the induction of apoptosis, 15-deoxy-PGJ2 increased the expression of proapoptotic proteins caspase 3, caspase 9, and Bax but down-regulated antiapoptotic protein Bcl-2. 15-Deoxy-PGJ2 also activated extracellular signal-regulated kinase (ERK) 2. In addition, mitogen-activated protein kinase kinase (MEK) ½ inhibitor PD98059 (2′-amino-3′-methoxyflavone) decreased 15-deoxy-PGJ2-induced ERK2 activation, and expression of PPAR-gamma, capase-3, and cyclin B1. Moreover, MEK½ inhibitor PD98059 significantly prevented against the 15-deoxy-PGJ2-induced cell growth inhibition. We also found that PPAR-gamma antagonist GW9662 (2-chloro-5-nitro-N-henylbenzamide) reversed the 15-deoxy-PGJ2-induced cell growth inhibition, PPAR-gamma expression, and activation of ERK2.

These results demonstrate that 15-deoxy-PGJ2 inhibits growth of human neuroblastoma cells via the induction of apoptosis in a PPAR-gamma-dependent manner through activation of ERK pathway and suggest that 15-deoxy-PGJ2 may have promising application as a therapeutic agent for neuroblastoma.

Eur J Pharmacol. 2003 April 18;466(3):225-34. “A non-thiazolidinedione partial peroxisome proliferator-activated receptor gamma ligand inhibits vascular smooth muscle cell growth.” Bruemmer D, Berger J P, Liu J, Kintscher U, Wakino S, Fleck E, Moller D E, Law R E. Division of Endocrinology, Diabetes and Hypertension, David Geffen School of Medicine, University of California-Los Angeles, Warren Hall, Suite 24-130, 900 Veteran Avenue, Los Angeles, Calif. 90095-7073, USA.

Several peroxisome proliferator-activated receptor gamma (PPARgamma) agonists of the thiazolidinedione class inhibit vascular smooth muscle cell proliferation. It is not known whether the antiproliferative activity of PPARgamma agonists is limited to the thiazolidinedione class and/or is directly mediated through PPARgamma-dependent transactivation of target genes.

We report here that a novel non-thiazolidinedione partial PPARgamma agonist (nTZDpa) attenuates rat aortic vascular smooth muscle cell proliferation. In a transfection assay for PPARgamma transcriptional activation, the non-thiazolidinedione partial PPARgamma agonist elicited approximately 25% of the maximal efficacy of the full PPARgamma agonist rosiglitazone. In the presence of the non-thiazolidinedione partial PPARgamma agonist, the transcriptional activity of the full agonist, rosiglitazone, was blunted, indicating that the non-thiazolidinedione partial PPARgamma agonist inhibits rosiglitazone-induced PPARgamma activity. The non-thiazolidinedione partial PPARgamma agonist (0.1-10 microM) inhibited vascular smooth muscle cell growth which was accompanied by an inhibition of retinoblastoma protein phosphorylation. Mitogen-induced downregulation of the cyclin-dependent kinase (CDK) inhibitor p27(kip1), and induction of the G1 cyclins cyclin D1, cyclin A, and cyclin E were also attenuated by the non-thiazolidinedione partial PPARgamma agonist. Maximal antiproliferative activity of the non-thiazolidinedione partial PPARgamma agonist required functional PPARgamma as adenovirus-mediated overexpression of a dominant-negative PPARgamma mutant partially reversed its inhibition of vascular smooth muscle cell growth. In contrast, overexpression of dominant-negative PPARgamma did not reverse the inhibitory effect of the non-thiazolidinedione partial PPARgamma agonist on cyclin D1. As the full PPARgamma agonist rosiglitazone exhibited no effect on cyclin D1, inhibition of that G1 cyclin by the non-thiazolidinedione partial PPARgamma agonist likely occurred through a PPARgamma-independent mechanism.

These data demonstrate that a non-thiazolidinedione partial PPARgamma agonist may constitute a novel therapeutic for proliferative vascular diseases and could provide additional evidence for the important role of PPARgamma in regulating vascular smooth muscle cell proliferation.

Br J Pharmacol. 2003 September;140(2):366-76. Epub 2003 Aug. 11 “Rosiglitazone and 15-deoxy-Delta12,14-prostaglandin J2, ligands of the peroxisome proliferator-activated receptor-gamma (PPAR-gamma), reduce ischaemia/reperfusion injury of the gut.” Cuzzocrea S, Pisano B, Dugo L, Ianaro A, Patel N S, Di Paola R, Genovese T, Chatterjee P K, Di Rosa M, Caputi A P, Thiemermann C. Department of Clinical and Experimental Medicine and Pharmacology, Torre Biologica, Policlinico Universitario, 98123 Messina, Italy.

The peroxisome proliferator-activated receptor-gamma (PPAR-gamma) is a member of the nuclear receptor superfamily of ligand-dependent transcription factors related to retinoid, steroid and thyroid hormone receptors. The thiazolidinedione rosiglitazone and the endogenous cyclopentenone prostaglandin (PG)D2 metabolite, 15-deoxy-Deltal 2,14-PGJ2 (15d-PGJ2), are two PPAR-gamma ligands, which modulate the transcription of target genes.

2. The aim of this study was to investigate the effect of rosiglitazone and 15d-PGJ2 on the tissue injury caused by ischaemia/reperfusion (I/R) of the gut.

3. I/R injury of the intestine was caused by clamping both the superior mesenteric artery and the coeliac trunk for 45 min, followed by release of the clamp allowing reperfusion for 2 or 4 h. This procedure results in splanchnic artery occlusion (SAO) shock.

4. Rats subjected to SAO developed a significant fall in mean arterial blood pressure, and only 10% of the animals survived for the entire 4 h reperfusion period. Surviving animals were killed for histological examination and biochemical studies. Rats subjected to SAO displayed a significant increase in tissue myeloperoxidase (MPO) activity and malondialdehyde (MDA) levels, significant increases in plasma tumour necrosis factor (TNF)-alpha and interleukin (IL)-1 beta levels and marked injury to the distal ileum.

5. Increased immunoreactivity to nitrotyrosine was observed in the ileum of rats subjected to SAO. Staining of sections of the ileum obtained from SAO rats with anti-intercellular adhesion molecule (ICAM-1) antibody resulted in diffuse staining.

6. Administration at 30 min prior to the onset of gut ischaemia of the two PPAR-gamma agonists (rosiglitazone (0.3 mg kg-1 i.v.) and 15d-PGJ2 (0.3 mg kg-1 i.v.)) significantly reduced the (i) fall in mean arterial blood pressure, (ii) mortality rate, (iii) infiltration of the reperfused intestine with polymorphonuclear neutrophils (MPO activity), (iv) lipid peroxidation (MDA levels), (v) production of proinflammatory cytokines (TNF-alpha and IL-1 beta) and (vi) histological evidence of gut injury. Administration of rosiglitazone and 15d-PGJ2 also markedly reduced the nitrotyrosine formation and the upregulation of ICAM-1 during reperfusion.

7. In order to elucidate whether the protective effects of rosiglitazone and 15d-PGJ2 are related to the activation of the PPAR-gamma receptor, we also investigated the effect of a PPAR-gamma antagonist, bisphenol A diglycidyl ether (BADGE), on the protective effects of rosiglitazone and 15d-PGJ2. BADGE (1 mg kg-1 administered i.v. 30 min prior to the treatment of rosiglitazone or 15d-PGJ2) significantly antagonised the effect of the two PPAR-gamma agonists and thus abolished the protective effect against gut I/R.

8. These results demonstrate that the two PPAR-gamma agonists, rosiglitazone and 15d-PGJ2, significantly reduce I/R injury of the intestine.

Eur J Pharmacol. 2004 Jan 1;483(1):79-93. “Rosiglitazone, a ligand of the peroxisome proliferator-activated receptor-gamma, reduces acute inflammation.” Cuzzocrea S, Pisano B, Dugo L, lanaro A, Maffia P, Patel N S, Di Paola R, lalenti A, Genovese T, Chatterjee P K, Di Rosa M, Caputi A P, Thiemermann C. Department of Clinical and Experimental Medicine and Pharmacology, School of Medicine, University of Messina, Torre Biologica, Policlinico Universitario Via C. Valeria, Gazzi, 98100 Messina, Italy.

Peroxisome proliferator-activated receptors (PPARs) are members of the nuclear hormone receptor superfamily of ligand-activated transcription factors that are related to retinoid, steroid and thyroid hormone receptors. The PPAR-gamma receptor subtype appears to play a pivotal role in the regulation of cellular proliferation and inflammation. The thiazolidinedione rosiglitazone (Avandia) is a peroxisome proliferator-activated receptor-gamma (PPAR-gamma) agonist that was recently approved by the Food and Drug Administration for treatment of type II diabetes mellitus.

In the present study, we have investigated the effects of rosiglitazone in animal models of acute inflammation (carrageenan-induced paw oedema and carrageenan-induced pleurisy). We report here for the first time that rosiglitazone (given at 1, 3 or 10 mg/kg i.p. concomitantly with carrageenan injection in the paw oedema model, or at 3, 10 or 30 mg/kg i.p. 15 min before carrageenan administration in the pleurisy model) exerts potent anti-inflammatory effects (e.g. inhibition of paw oedema, pleural exudate formation, mononuclear cell infiltration and histological injury) in vivo. Furthermore, rosiglitazone reduced: (1) the increase in the staining (immunohistochemistry) for nitrotyrosine and poly (ADP-ribose) polymerase (PARP), (2) the expression of inducible nitric oxide synthase (iNOS), cyclooxygenase-2 (COX-2), intercellular adhesion molecules-1 (ICAM-1) and P-selectin in the lungs of carrageenan-treated rats.

In order to elucidate whether the protective effect of rosiglitazone is related to activation of the PPAR-gamma receptor, we also investigated the effect of a PPAR-gamma antagonist, bisphenol A diglycidyl ether (BADGE), on the protective effects of rosiglitazone. BADGE (30 mg/kg i.p.) administered 30 min prior to treatment with rosiglitazone significantly antagonized the effect of the PPAR-gamma agonist and thus abolished the anti-inflammatory effects of rosiglitazone. We propose that rosiglitazone and other potent PPAR-gamma agonists may be useful in the therapy of inflammation.

J Biol. Chem. 2000 Nov. 17;275(46):35715-22. “Peroxisome proliferator-activated receptors and hepatic stellate cell activation.” Miyahara T, Schrum L, Rippe R, Xiong S, Yee H F Jr, Motomura K, Anania F A, Willson T M, Tsukamoto H. Departments of Medicine and Pathology, Keck School of Medicine of the University of Southern California, Los Angeles, Calif. 90033, USA.

The present study examined the roles of peroxisome proliferator-activated receptors (PPAR) in activation of hepatic stellate cells (HSC), a pivotal event in liver fibrogenesis. RNase protection assay detected mRNA for PPARgammal but not that for the adipocyte-specific gamma2 isoform in HSC isolated from sham-operated rats, whereas the transcripts for neither isoforms were detectable in HSC from cholestatic liver fibrosis induced by bile duct ligation (BDL). Semi-quantitative reverse transcriptase-polymerase chain reaction confirmed a 70% reduction in PPARgamma mRNA level in HSC from BDL. Nuclear extracts from BDL cells showed an expected diminution of binding to PPAR-responsive element, whereas NF-kappaB and AP-1 binding were increased. Treatment of cultured-activated HSC with ligands for PPARgamma (10 microm 15-deoxy-Delta(12,14)-PGJ(2) (15dPGJ(2)); 0.1 approximately 10 microm BRL49653) inhibited DNA and collagen synthesis without affecting the cell viability. Suppression of HSC collagen by 15dPGJ(2) was abrogated 70% by the concomitant treatment with a PPARgamma antagonist (GW9662).

HSC DNA and collagen synthesis were inhibited by WY14643 at the concentrations known to activate both PPARalpha and gamma (>100 microm) but not at those that only activate PPARalpha (<10 microm) or by a synthetic PPARalpha-selective agonist (GW9578). 15dPGJ(2) reduced alpha1 (I) procollagen, smooth muscle alpha-actin, and monocyte chemotactic protein-1 mRNA levels while inducing matrix metalloproteinase-3 and CD36. 15dPGJ(2) and BRL49653 inhibited alpha1 (I) procollagen promoter activity. Tumor necrosis factor alpha (10 ng/ml) reduced PPARgamma mRNA, and this effect was prevented by the treatment with 15dPGJ(2).

These results demonstrate that HSC activation is associated with the reductions in PPARgamma expression and PPAR-responsive element binding in vivo and is reversed by the treatment with PPARgamma ligands in vitro. These findings implicate diminished PPARgamma signaling in molecular mechanisms underlying activation of HSC in liver fibrogenesis and the potential therapeutic value of PPARgamma ligands for liver fibrosis.

All PPARs are, albeit to different extents, activated by fatty acids and derivatives; PPAR-alpha binds the hypolipidemic fibrates whereas antidiabetic glitazones are ligands for PPAR-gamma. PPAR-alpha activation mediates pleiotropic effects such as stimulation of lipid oxidation, alteration in lipoprotein metabolism and inhibition of vascular inflammation. PPAR-alpha activators increase hepatic uptake and the esterification of free fatty acids by stimulating the fatty acid transport protein and acyl-CoA synthetase expression. In skeletal muscle and heart, PPAR-alpha increases mitochondrial free fatty acid uptake and the resulting free fatty acid oxidation through stimulating the muscle-type carnitine palmitoyltransferase-I. The effect of fibrates on the metabolism of triglyceride-rich lipoproteins is due to a PPAR-alpha dependent stimulation of lipoprotein lipase and an inhibition of apolipoprotein C-III expressions, whereas the increase in plasma HDL cholesterol depends on an overexpression of apolipoprotein A-I and apolipoprotein A-II. PPARs are also expressed in atherosclerotic lesions.

PPAR-alpha is present in endothelial and smooth muscle cells, monocytes and monocyte-derived macrophages. It inhibits inducible nitric oxide synthase in macrophages and prevents the IL-1-induced expression of IL-6 and cyclooxygenase-2, as well as thrombin-induced endothelin-i expression, as a result of a negative transcriptional regulation of the nuclear factor-kappa B and activator protein-1 signalling pathways. PPAR activation also induces apoptosis in human monocyte-derived macrophages most likely through inhibition of nuclear factor-kappa B activity. Therefore, the pleiotropic effects of PPAR-alpha activators on the plasma lipid profile and vascular wall inflammation certainly participate in the inhibition of atherosclerosis development observed in angiographically documented intervention trials with fibrates.

Impaired skin wound healing in peroxisome proliferator-activated receptor (PPAR) and PPARβ mutant mice. Liliane Michalik1, Béatrice Desvergne1, Nguan Soon Tan1, Sharmila Basu-Modak1, Pascal Escher1, Jennifer Rieusset1, Jeffrey M. Peters3, Gürkan Kaya2, Frank J. Gonzalez3, Jozsef Zakany4, Daniel Metzger5, Pierre Chambon5, Denis Duboule4 and Walter Wahli1

(1) Institut de Biologie Animale, Universite de Lausanne, Bâtiment de Biologie, CH-1015 Lausanne, Switzerland

(2) Department of Dermatology, University Hospital of Geneva, CH-1212 Geneva, Switzerland

(3) Laboratory of Molecular Carcinogenesis, National Cancer Institute, National Institute of Health, Bethesda, Md. 20892

(4) Département de Zoologie, Université de Genéve, Sciences III, CH-1211 Geneva 4, Switzerland

(5) Institut de Génétique et de Biologie Moléculaire et Cellulaire, Centre National de la Recherche Scientifique/Institut National de la Santé et de la Recherche Médicale/ULP/Collége de France, 67404 llikirch Cedex, CU de Strasbourg, France

We show here that the, β, and isotypes of peroxisome proliferator-activated receptor (PPAR) are expressed in the mouse epidermis during fetal development and that they disappear progressively from the interfollicular epithelium after birth. Interestingly, PPAR and β expression is reactivated in the adult epidermis after various stimuli, resulting in keratinocyte proliferation and differentiation such as tetradecanoylphorbol acetate topical application, hair plucking, or skin wound healing. Using PPAR, β, and mutant mice, we demonstrate that PPAR and β are important for the rapid epithelialization of a skin wound and that each of them plays a specific role in this process. PPAR is mainly involved in the early inflammation phase of the healing, whereas PPARβ is implicated in the control of keratinocyte proliferation.

In addition and very interestingly, PPARβ mutant primary keratinocytes show impaired adhesion and migration properties. Thus, the findings presented here reveal unpredicted roles for PPAR and β in adult mouse epidermal repair.

It is obvious that alternative or adjunctive therapy is needed for persons infected with HIV.

It is rational to support PARP-1 activation with the concomitant ablation of the targeted virus.

Thus it is logical and valuable to administer a molecule that stimulates ATP, NAD and the Krebs cycle (Tricarboxylic acid cycle) downstream of the glycolytic blockade in order to support PARP-1 ablation with concomitant modulation of PPAR to reduce adverse effects seen from the combination therapy in the treatment of HIV.

Support, rather than inhibition of PARP-1, thereby inducing ablation of viral infections and genomic errors with Methyl Pyruvate and its supra-normal stimulation of the Krebs cycle downstream of glycolosis while simultaneously activating PPAR is indeed original.

Any pharmacologically acceptable salt can be used, provided that it is suitable and practical for administration to humans, sufficiently stable under reasonable storage conditions to have an adequate shelf life, and physiologically acceptable when introduced into the body by a suitable route of administration. The nature of the salt is not critical, provided that it is non-toxic and does not substantially interfere with the desired activity.

### SUMMARY OF THE INVENTION

The present invention pertains to methods of increasing cellular energy production downstream from and independently of glycolosis for an individual afflicted with a viral infection or event that induces continuous chronic or acute PARP-1 activation. Such a viral infection or event can be ameliorated by administering to the afflicted individual an amount of methyl pyruvate sufficient to protect against cellular ATP and NAD depletion thereby supporting PARP-1 in preventing, reducing or ameliorating the symptoms. Typical dosages of a methyl pyruvate will depend on factors such as size, age, health, the virus strain/disease/event and duration of the virus strain/disease/event. This treatment is effective when administered on a chronic or acute basis.

A preferred mode of use involves co-administration of methyl pyruvate compounds along with one or more agents that promote energy.

A preferred mode of use involves co-administration of methyl pyruvate compounds along with one or more agents that promote proper mitochondria function while decreasing oxidative stress.

The present invention further pertains to methods of use of methyl pyruvate compounds in combination with vitamins, coenzymes, mineral substances, amino acids, antioxidants, herbs, and creatine compounds, or pharmaceutical drugs which act on the cell for enhancing function and viability.

Compounds effective for this purpose include the present invention, which also provides compositions containing methyl pyruvate compounds in combination with a pharmaceutically acceptable carrier, and effective amounts of other agents, which act, to prophylactically and/or therapeutically treat a subject with a viral infection or for an event that induces PARP-1 activation and concomitant depletion of ATP and NAD.

Some of the diseases susceptible to treatment with methyl pyruvate compounds according to the present invention include, but are not limited to HIV-1, Hepatitis C, Genital Warts, Influenza, Herpes Simplex, Common Cold, Rubella, Rabies, Severe Acute Respiratory Syndrome, Hantavirus Infections, Alzheimer disease, Parkinson's disease, Huntington's disease, motor neuron disease, diabetic and toxic neuropathies, traumatic nerve injury, multiple sclerosis, acute disseminated encephalomyelitis, acute necrotizing hemorrhagic leukoencephalitis, diseases of dysmyelination, mitochondrial diseases, fungal and bacterial infections, migrainous disorders, stroke, aging, dementia, and mental disorders such as depression and schizophrenia. Any disease, viral infection or event caused by impairment of intracellular energy metabolism or that depletes available ATP, NAD, especially if the impairment were in the Krebs cycle or the affliction induces chronic or acute PARP-1 activation, methyl pyruvate could be administered orally or infused on a chronic or acute basis to maintain cellular energy at a level that will support PARP-1 activation and the concomitant ablation or amelioration of the diease, infection or event.

The present invention further pertains to methods of use of methyl pyruvate compounds in treatment to protect against ATP, NAD depletion due to ischemia (inadequate blood flow, which can be caused by stroke, cardiac arrest, or other events) or due to hypoxia, hypoglycemia, or cellular disorders which interfere with the energy metabolism of cells can be effective when administered before (pre-coditioning) or after the onset of an event that triggers acute ATP, NAD depletion or PARP-1 activation. Use of methyl pyruvate can be effective when administered orally or infused on an acute basis. Typical dosages of methyl pyruvate compounds will depend on factors such as the size and condition of the patient and the amount of time that has elapsed since the onset of the ischemic event.

### DESCRIPTION OF THE PREFERRED EMBODIMENTS

This invention entails a use of methyl pyruvate to increase cellular energy production to allow continuous PARP activation without the concomitant depletion of ATP, NAD and necrotic cell death. Methyl pruvate is the ionized form of methyl pyruvic acid (CH3C(O)CO2CH3). At physiologic pH, the hydrogen proton dissociates from the carboxylic acid group, thereby generating the methyl pyruvate anion. When used as a pharmaceutical or dietary supplement, this anion can be formulated as a salt, using a monovalent or divalent cation such as sodium, potassium, magnesium, or calcium.

Pancreatic Beta-Cell as a Model

The energy requirements of most cells supplied with glucose are fulfilled by glycolytic and oxidative metabolism, yielding ATP. When cytosolic and mitochondrial contents in ATP, ADP and AMP were measured in islets incubated for 45 min at increasing concentrations of D-glucose and then exposed for 20 s to digitonin. The latter treatment failed to affect the total islet ATP/ADP ratio and adenylate charge. D-Glucose caused a much greater increase in cytosolic than mitochondrial ATP/ADP ratio. In the cytosol, a sigmoidal pattern characterized the changes in ATP/ADP ratio at increasing concentrations of D-glucose. These findings are compatible with the view that cytosolic ATP participates in the coupling of metabolic to ionic events in the process of nutrient-induced insulin release.

To gain insight into the regulation of pancreatic beta-cell mitochondrial metabolism, the direct effects on respiration of different mitochondrial substrates, variations in the ATP/ADP ratio and free Ca2+ were examined using isolated mitochondria and permeabilized clonal pancreatic beta-cells (HIT). Respiration from pyruvate was high and not influenced by Ca2+ in State 3 or under various redox states and fixed values of the ATP/ADP ratio; nevertheless, high Ca2+ elevated pyridine nucleotide fluorescence, indicating activation of pyruvate dehydrogenase by Ca2+.

Furthermore, in the presence of pyruvate, elevated Ca2+ stimulated CO2 production from pyruvate, increased citrate production and efflux from the mitochondria and inhibited CO2 production from palmitate. The latter observation suggests that beta-cell fatty acid oxidation is not regulated exclusively by malonyl-CoA but also by the mitochondrial redox state. alpha-Glycerophosphate (alpha-GP) oxidation was Ca(2+)-dependent with a half-maximal rate observed at around 300 nM Ca2+. It was recently demonstrated that increases in respiration precede increases in Ca2+ in glucose-stimulated clonal pancreatic beta-cells (HIT), indicating that Ca2+ is not responsible for the initial stimulation of respiration. It is suggested that respiration is stimulated by increased substrate (alpha-GP and pyruvate) supply together with oscillatory increases in ADP. The rise in Ca2+, which in itself may not significantly increase net respiration, could have the important functions of
(1) activating the alpha-GP shuttle, to maintain an oxidized cytosol and high glycolytic flux;
   (2) activating pyruvate dehydrogenase, and indirectly pyruvate carboxylase, to sustain production of citrate and hence the putative signal coupling factors, malonyl-CoA and acyl-CoA;
   (3) increasing mitochondrial redox state to implement the switch from fatty acid to pyruvate oxidation.

Glucose-stimulated increases in mitochondrial metabolism are generally thought to be important for the activation of insulin secretion. Pyruvate dehydrogenase (PDH) is a key regulatory enzyme, believed to govern the rate of pyruvate entry into the citrate cycle. It has been shown that elevated glucose concentrations (16 or 30 vs 3 mM) cause an increase in PDH activity in both isolated rat islets, and in a clonal beta-cell line (MIN6).

However, increases in PDH activity elicited with either dichloroacetate, or by adenoviral expression of the catalytic subunit of pyruvate dehydrogenase phosphatase, were without effect on glucose-induced increases in mitochondrial pyridine nucleotide levels, or cytosolic ATP concentration, in MIN6 cells, and insulin secretion from isolated rat islets. Similarly, the above parameters were unaffected by blockade of the glucose-induced increase in PDH activity by adenovirus-mediated over-expression of PDH kinase (PDK). Thus, activation of the PDH complex plays an unexpectedly minor role in stimulating glucose metabolism and in triggering insulin release.

In pancreatic beta-cells, a rise in cytosolic ATP is also a critical signaling event, coupling closure of ATP-sensitive K+ channels (KATP) to insulin secretion via depolarization-driven increases in intracellular Ca2+. Glycolytic but not Krebs cycle metabolism of glucose is critically involved in this signaling process.

While inhibitors of glycolysis suppressed glucose-stimulated insulin secretion, blockers of pyruvate transport or Krebs cycle enzymes were without effect. While pyruvate was metabolized in islets to the same extent as glucose, it produced no stimulation of insulin secretion and did not block KATP.

In pancreatic beta-cells, methyl pyruvate is a potent secretagogue and is widely used to study stimulus-secretion coupling. MP stimulated insulin secretion in the absence of glucose, with maximal effect at 5 mM. MP depolarized the beta-cell in a concentration-dependent manner (5-20 mM). Pyruvate failed to initiate insulin release (5-20 mM) or to depolarize the membrane potential. ATP production in isolated beta-cell mitochondria was detected as accumulation of ATP in the medium during incubation in the presence of malate or glutamate in combination with pyruvate or MP. ATP production by MP and glutamate was higher than that induced by pyruvate/glutamate. Pyruvate (5 mM) or MP (5 mM) had no effect on the ATP/ADP ratio in whole islets, whereas glucose (20 mM) significantly increased the whole islet ATP/ADP ratio.

In contrast with pyruvate, which barely stimulates insulin secretion, methyl pyruvate was suggested to act as an effective mitochondrial substrate. Methyl pyruvate elicited electrical activity in the presence of 0.5 mM glucose, in contrast with pyruvate. Accordingly, methyl pyruvate increased the cytosolic free Ca(2+) concentration after an initial decrease, similar to glucose. However, in contrast with glucose, methyl pyruvate even slightly decreased NAD(P)H autofluorescence and did not influence ATP production or the ATP/ADP ratio. Therefore, MP-induced beta-cell membrane depolarization or insulin release does not relate directly to mitochondrial ATP production.

The finding that methyl pyruvate directly inhibited a cation current across the inner membrane of Jurkat T-lymphocyte mitochondria suggests that this metabolite may increase ATP production in beta-cells by activating the respiratory chains without providing reduction equivalents. This mechanism may account for a slight and transient increase in ATP production. Furthermore methyl pyruvate inhibited the K(ATP) current measured in the standard whole-cell configuration. Accordingly, single-channel currents in inside-out patches were blocked by methyl pyruvate. Therefore, the inhibition of K(ATP) channels, and not activation of metabolism, mediates the induction of electrical activity in pancreatic beta-cells by methyl pyruvate.

As a membrane-permeant analog, methyl pyruvate, produced a block of KATP, a sustained rise in [Ca2+]i, and an increase in insulin secretion 6-fold the magnitude of that induced by glucose. This indicates that ATP derived from mitochondrial pyruvate metabolism does not substantially contribute to the regulation of KATP responses to a glucose challenge. Supporting the notion of sub-compartmentation of ATP within the beta-cell. Supra-normal stimulation of the Krebs cycle by methyl pyruvate can, however, overwhelm intracellular partitioning of ATP and thereby drive insulin secretion.

The metabolism of methyl pyruvate was compared to that of pyruvate in isolated rat pancreatic islets. Methyl pyruvate was found to be more efficient than pyruvate in supporting the intramitochondrial conversion of pyruvate metabolites to amino acids, inhibiting D-[5-3H]glucose utilization, maintaining a high ratio between D-[3,4-14C] glucose or D-[6-14C]glucose oxidation and D-[5-3H]glucose utilization, inhibiting the intramitochondrial conversion of glucose-derived 2-keto acids to their corresponding amino acids, and augmenting 14CO2 output from islets prelabeled with L-[U-14C] glutamine.

Methyl pyruvate also apparently caused a more marked mitochondrial alkalinization than pyruvate, as judged from comparisons of pH measurements based on the use of either a fluorescein probe or 14C-labeled 5,5-dimethyl-oxazolidine-2,4-dione. Inversely, pyruvate was more efficient than methyl pyruvate in increasing lactate output and generating L-alanine. These converging findings indicate that, by comparison with exogenous pyruvate, its methyl ester is preferentially metabolized in the mitochondrial, rather than cytosolic, domain of islet cells. It is proposed that both the positive and the negative components of methyl pyruvate insulinotropic action are linked to changes in the net generation of reducing equivalents, ATP and H+.

Methyl pyruvate was found to exert a dual effect on insulin release from isolated rat pancreatic islets. A positive insulinotropic action prevailed at low concentrations of D-glucose, in the 2.8 to 8.3 mM range, and at concentrations of the ester not exceeding 1 0.0 mM. It displayed features typical of a process of nutrient-stimulated insulin release, such as decreased K+ conductance, enhanced Ca2+ influx, and stimulation of proinsulin biosynthesis. A negative insulinotropic action of methyl pyruvate was also observed, however, at a high concentration of D-glucose (16.7 mM) and/or at a high concentration of the methyl ester (20.0 mM). It was apparently not attributable to any adverse effect of methyl pyruvate on ATP generation, but might be due to hyperpolarization of the plasma membrane. The ionic determinant(s) of the latter change was not identified. The dual effect of methyl pyruvate probably accounts for an unusual time course of the secretory response, including a dramatic and paradoxical stimulation of insulin release upon removal of the ester.

Pancreatic beta-cell metabolism was followed during glucose and pyruvate stimulation of pancreatic islets using quantitative two-photon NAD(P)H imaging. The observed redox changes, spatially separated between the cytoplasm and mitochondria, were compared with whole islet insulin secretion.

As expected, both NAD(P)H and insulin secretion showed sustained increases in response to glucose stimulation. In contrast, pyruvate caused a much lower NAD(P)H response and did not generate insulin secretion. Low pyruvate concentrations decreased cytoplasmic NAD(P)H without affecting mitochondrial NAD(P)H, whereas higher concentrations increased cytoplasmic and mitochondrial levels. However, the pyruvate-stimulated mitochondrial increase was transient and equilibrated to near-base-line levels. Inhibitors of the mitochondrial pyruvate-transporter and malate-aspartate shuttle were utilized to resolve the glucose- and pyruvate-stimulated NAD(P)H response mechanisms.

These data showed that glucose-stimulated mitochondrial NAD(P)H and insulin secretion are independent of pyruvate transport but dependent on NAD(P)H shuttling. In contrast, the pyruvate-stimulated cytoplasmic NAD(P)H response was enhanced by both inhibitors. Surprisingly the malate-aspartate shuttle inhibitor enabled pyruvate-stimulated insulin secretion. These data support a model in which glycolysis plays a dominant role in glucose-stimulated insulin secretion. Based on these data, it was proposed as a mechanism for glucose-stimulated insulin secretion that includes allosteric inhibition of tricarboxylic acid cycle enzymes and pH dependence of mitochondrial pyruvate transport.

Pyridine dinucleotides (NAD and NADP) are ubiquitous cofactors involved in hundreds of redox reactions essential for the energy transduction and metabolism in all living cells. In addition, NAD also serves as a substrate for ADP-ribosylation of a number of nuclear proteins, for silent information regulator 2 (Sir2)-like histone deacetylase that is involved in gene silencing regulation, and for cyclic ADP ribose (cADPR)-dependent Ca(2+) signaling. Pyridine nucleotide adenylyltransferase (PNAT) is an indispensable central enzyme in the NAD biosynthesis pathways catalyzing the condensation of pyridine mononucleotide (NMN or NaMN) with the AMP moiety of ATP to form NAD (or NaAD).

In isolated pancreatic islets, pyruvate causes a shift to the left of the sigmoidal curve relating the rate of insulin release to the ambient glucose concentration. The magnitude of this effect is related to the concentration of pyruvate (5--90 mM) and, at a 30 mM concentration, is equivalent to that evoked by 2 mM-glucose.
(1) 2. In the presence of glucose 8 mM), the secretory response to pyruvate is an immediate process, displaying a biphasic pattern.
   (2)3. The insulinotropic action of pyruvate coincides with an inhibition of 45Ca efflux and a stimulation of 45Ca net uptake. The relationship between 45Ca uptake and insulin release displays its usual pattern in the presence of pyruvate.
   (3)4. Exogenous pyruvate rapidly accumulates in the islets in amounts close to those derived from the metabolism of glucose. The oxidation of [2-14C]pyruvate represents 64% of the rate of [1-14C]pyruvate decarboxylation and, at a 30 mM concentration, is comparable with that of 8 mM-[U-14C]glucose.
   (4)5. When corrected for the conversion of pyruvate into lactate, the oxidation of 30 mM-pyruvate corresponds to a net generation of about 314 pmol of reducing equivalents/120 min per islet.
   (5)6. Pyruvate does not affect the rate of glycolysis, but inhibits the oxidation of glucose. Glucose does not affect pyruvate oxidation.
   (6)7. Pyruvate (30 mM) does not affect the concentration of ATP, ADP and AMP in the islet cells.
   (7)8. Pyruvate (30 mM) increases the concentration of reduced nicotinamide nucleotides in the presence but not in the absence of glucose. A close correlation is seen between the concentration of reduced nicotinamide nucleotides and the net uptake of 45 Ca.
   (8)9. Pyruvate, like glucose, modestly stimulates lipogenesis.
   (9)10. Pyruvate, in contrast with glucose, markedly inhibits the oxidation of endogenous nutrients. The latter effect accounts for the apparent discrepancy between the rate of pyruvate oxidation and the magnitude of its insulinotropic action.
   (10) 11. It is concluded that the effect of pyruvate to stimulate insulin release depends on its ability to increase the concentration of reduced nicotinamide nucleotides in the islet cells.

Glucose-stimulated insulin secretion is a multi-step process dependent on cell metabolic flux. Previous studies on intact pancreatic islets used two-photon NAD(P)H imaging as a quantitative measure of the combined redox signal from NADH and NADPH (referred to as NAD(P)H). These studies showed that pyruvate, a non-secretagogue, enters -cells and causes a transient rise in NAD(P)H. To further characterize the metabolic fate of pyruvate, a one-photon flavoprotein microscopy has been developed as a simultaneous assay of lipoamide dehydrogenase (LipDH) autofluorescence. This flavoprotein is in direct equilibrium with mitochondrial NADH.

Using this method, the glucose-dose response is consistent with an increase in both NADH and NADPH. In contrast, the transient rise in NAD(P)H observed with pyruvate stimulation is not accompanied by a significant change in LipDH, which indicates that pyruvate raises cellular NADPH without raising NADH. In comparison, methyl pyruvate stimulated a robust NADH and NADPH response. These data provide new evidence that exogenous pyruvate does not induce a significant rise in mitochondrial NADH. This inability likely results in its failure to produce the ATP necessary for stimulated secretion of insulin. Overall, these data are consistent with either restricted PDH dependent metabolism or a buffering of the NADH response by other metabolic mechanisms.

Glucose metabolism in glycolysis and in mitochondria is pivotal to glucose-induced insulin secretion from pancreatic beta cells. One or more factors derived from glycolysis other than pyruvate appear to be required for the generation of mitochondrial signals that lead to insulin secretion. The electrons of the glycolysis-derived reduced form of nicotinamide adenine dinucleotide (NADH) are transferred to mitochondria through the NADH shuttle system. By abolishing the NADH shuttle function, glucose-induced increases in NADH autofluorescence, mitochondrial membrane potential, and adenosine triphosphate content were reduced and glucose-induced insulin secretion was abrogated. The NADH shuttle evidently couples glycolysis with activation of mitochondrial energy metabolism to trigger insulin secretion.

To determine the role of the NADH shuttle system composed of the glycerol phosphate shuttle and malate-aspartate shuttle in glucose-induced insulin secretion from pancreatic beta cells, mice which lack mitochondrial glycerol-3 phosphate dehydrogenase mGPDH), a rate-limiting enzyme of the glycerol phosphate shuttle were used. When both shuttles were halted in mGPDH-deficient islets treated with aminooxyacetate, an inhibitor of the malate-aspartate shuttle, glucose-induced insulin secretion was almost completely abrogated. Under these conditions, although the flux of glycolysis and supply of glucose-derived pyruvate into mitochondria were unaffected, glucose-induced increases in NAD(P)H autofluorescence, mitochondrial membrane potential, Ca2+ entry into mitochondria, and ATP content were severely attenuated.

This study provides the first direct evidence that the NADH shuttle system is essential for coupling glycolysis with the activation of mitochondrial energy metabolism to trigger glucose-induced insulin secretion and thus revises the classical model for the metabolic signals of glucose-induced insulin secretion.

Incubation of porcine carotid arteries with 0.4 mmol amino-oxyacetic acid an inhibitor of glutamate-oxaloacetate transaminase and, hence the malate-aspartate shuttle, inhibited O2 consumption by 21%, decreased the content of phosphocreatine and inhibited activity of the tricarboxylic acid cycle. The rate of glycolysis and lactate production was increased but glucose oxidation was inhibited. These effects of amino-oxyacetic acid were accompanied by evidence of inhibition of the malate-aspartate shuttle and elevation in the cytoplasmic redox potential and NADH/NAD ratio as indicated by elevation of the concentration ratios of the lactate/pyruvate and glycerol-3-phosphate/dihydroxyacetone phosphate metabolite redox couples. Addition of the fatty acid octanoate normalized the adverse energetic effects of malate-aspartate shuttle inhibition.

It is concluded that the malate-aspartate shuttle is a primary mode of clearance of NADH reducing equivalents from the cytoplasm in vascular smooth muscle. Glucose oxidation and lactate production are influenced by the activity of the shuttle. The results support the hypothesis that an increased cytoplasmic NADH redox potential impairs mitochondrial energy metabolism.

Beta-Methyleneaspartate, a specific inhibitor of aspartate aminotransferase (EC 2.6.1.1.), was used to investigate the role of the malate-aspartate shuttle in rat brain synaptosomes. Incubation of rat brain cytosol, “free” mitochondria, synaptosol, and synaptic mitochondria, with 2 mM beta-methyleneaspartate resulted in inhibition of aspartate aminotransferase by 69%, 67%, 49%, and 76%, respectively. The reconstituted malate-aspartate shuttle of “free” brain mitochondria was inhibited by a similar degree (53%). As a consequence of the inhibition of the aspartate aminotransferase, and hence the malate-aspartate shuttle, the following changes were observed in synaptosomes: decreased glucose oxidation via the pyruvate dehydrogenase reaction and the tricarboxylic acid cycle; decreased acetylcholine synthesis; and an increase in the cytosolic redox state, as measured by the lactate/pyruvate ratio.

The main reason for these changes can be attributed to decreased carbon flow through the tricarboxylic acid cycle (i.e., decreased formation of oxaloacetate), rather than as a direct consequence of changes in the NAD+/NADH ratio. Malate/glutamate oxidation in “free” mitochondria was also decreased in the presence of 2 mM beta-methyleneaspartate. This is probably a result of decreased glutamate transport into mitochondria as a result of low levels of aspartate, which are needed for the exchange with glutamate by the energy-dependent glutamate-aspartate translocator.

Aminooxyacetate, an inhibitor of pyridoxal-dependent enzymes, is routinely used to inhibit gamma-aminobutyrate metabolism. The bioenergetic effects of the inhibitor on guinea-pig cerebral cortical synaptosomes are investigated. It prevents the reoxidation of cytosolic NADH by the mitochondria by inhibiting the malate-aspartate shuttle, causing a 26 mV negative shift in the cytosolic NAD+/NADH redox potential, an increase in the lactate/pyruvate ratio and an inhibition of the ability of the mitochondria to utilize glycolytic pyruvate. The 3-hydroxybutyrate/acetoacetate ratio decreased significantly, indicating oxidation of the mitochondrial NAD+/NADH couple.

The results are consistent with a predominant role of the malate-aspartate shuttle in the reoxidation of cytosolic NADH in isolated nerve terminals. Aminooxyacetate limits respiratory capacity and lowers mitochondrial membrane potential and synaptosomal ATP/ADP ratios to an extent similar to glucose deprivation.

Variations in the cytoplasmic redox potential (Eh) and NADH/NAD ratio as determined by the ratio of reduced to oxidized intracellular metabolite redox couples may affect mitochondrial energetics and alter the excitability and contractile reactivity of vascular smooth muscle.

To test these hypotheses, the cytoplasmic redox state was experimentally manipulated by incubating porcine carotid artery strips in various substrates. The redox potentials of the metabolite couples [lactate]/[pyruvate]i and [glycerol 3-phosphate]/[dihydroxyacetone phosphate]i varied linearly (r=0.945), indicating equilibrium between the two cytoplasmic redox systems and with cytoplasmic NADH/NAD. Incubation in physiological salt solution (PSS) containing 10 mm pyruvate ([lact]/[pyr]=0.6) increased O2 consumption approximately 45% and produced anaplerosis of the tricarboxylic acid (TCA cycle), whereas incubation with 10 mm lactate-PSS ([lact]/[pyr]i=47) was without effect. A hyperpolarizing dose of external KCl (10 mM) produced a decrease in resting tone of muscles incubated in either glucose-PSS (−0.8+/−0.8 g) or pyruvate-PSS (−2.1+/−0.8 g), but increased contraction in lactate-PSS (1.5+/−0.7 g) (n=12-18, P<0.05). The rate and magnitude of contraction with 80 mm KCl (depolarizing) was decreased in lactate-PSS(P=0.001). Slopes of KCl concentration-response curves indicated pyruvate>glucose>lactate (P<0.0001); EC50 in lactate (29.1+/−1.0 mM) was less than that in either glucose (32.1+/−0.9 mm) or pyruvate (32.2+/−1.0 mM), P<0.03.

The results are consistent with an effect of the cytoplasmic redox potential to influence the excitability of the smooth muscle and to affect mitochondrial energetics.

The cytoplasmic NADH/NAD redox potential affects energy metabolism and contractile reactivity of vascular smooth muscle. NADH/NAD redox state in the cytosol is predominately determined by glycolysis, which in smooth muscle is separated into two functionally independent cytoplasmic compartments, one of which fuels the activity of Na(+)-K(+)-ATPase. The effect was examined of varying the glycolytic compartments on cystosolic NADH/NAD redox state. Inhibition of Na(+)-K(+)-ATPase by 10 microM ouabain resulted in decreased glycolysis and lactate production. Despite this, intracellular concentrations of the glycolytic metabolite redox couples of lactate/pyruvate and glycerol-3-phosphate/dihydroxyacetone phosphate (thus NADH/NAD) and the cytoplasmic redox state were unchanged. The constant concentration of the metabolite redox couples and redox potential was attributed to
(1) decreased efflux of lactate and pyruvate due to decreased activity of monocarboxylate B—H(+) transporter secondary to decreased availability of H(+) for cotransport and
(2) increased uptake of lactate (and perhaps pyruvate) from the extracellular space, probably mediated by the monocarboxylate-H(+) transporter, which was specifically linked to reduced activity of Na(+)-K(+)-ATPase.

It was concluded that redox potentials of the two glycolytic compartments of the cytosol maintain equilibrium and that the cytoplasmic NADH/NAD redox potential remains constant in the steady state despite varying glycolytic flux in the cytosolic compartment for Na(+)-K(+)-ATPase.

Methyl Pyruvate as a PPAR Agonist

Peroxisomal proliferator-activated receptors (PPARs) belong to a nuclear receptor superfamily of ligand-activated transcription factors. Peroxisome proliferator-activated receptor (PPAR) is activated when a ligand binds to the ligand-binding domain at the side of C-termini.

So far, three types of isoforms of alpha form, gamma form and delta form have been identified as PPARs, and the expression tissues and the functions are different respectively. Peroxisome proliferators are a structurally diverse group of compounds which, when administered to rodents, elicit dramatic increases in the size and number of hepatic and renal peroxisomes, as well as concomitant increases in the capacity of peroxisomes to metabolize fatty acids via increased expression of the enzymes required for the beta-oxidation cycle It is known that the alpha-isoform of peroxisome proliferator-activated receptor (PPAR.alpha) acts to stimulate peroxisomal proliferation in the rodent liver which leads to enhanced fatty oxidation by this organ. (PPAR) alpha is a nuclear receptor that is mainly expressed in tissues with a high degree of fatty acid oxidation such as liver, heart, and skeletal muscle.

There is a sex difference in PPARalpha expression. Male rats have higher levels of hepatic PPARalpha mRNA and protein than female rats. Chemicals included in this group are the fibrate class of hypolipidermic drugs, herbicides, and phthalate plasticizers. Peroxisome proliferation can also be elicited by dietary or physiological factors such as a high-fat diet and cold acclimatization. The importance of peroxisomes in humans is stressed by the existence of a group of genetic diseases in man in which one or more peroxisomal functions are impaired. Most of the functions known to take place in peroxisomes have to do with lipids. Indeed, peroxisomes are capable of 1. fatty acid beta-oxidation 2. fatty acid alpha-oxidation 3. synthesis of cholesterol and other isoprenoids 4. ether-phospholipid synthesis and 5. biosynthesis of polyunsaturated fatty acids.

In animal cells peroxisomes as well as mitochondria are capable of degrading lipids via beta-oxidation. Nevertheless, there are important differences between the two systems.
(1) The peroxisomal and mitochondrial beta-oxidation enzymes are different proteins.
   (2) Peroxisomal beta-oxidation does not degrade fatty acids completely but acts as a chain-shortening system, catalyzing only a limited number of beta-oxidation cycles.
   (3) Peroxisomal beta-oxidation is not coupled to oxidative phosphorylation and is thus less efficient than mitochondrial beta-oxidation as far as energy conservation is concerned.
   (4) Peroxisomal beta-oxidation is not regulated by malonyl-CoA and—as a consequence—by feeding as opposed to starvation.

Insight into the mechanism whereby peroxisome proliferators exert their pleiotropic effects was provided by the identification of a member of the nuclear hormone receptor superfamily activated by these chemicals. This receptor, termed peroxisome proliferator activated receptor alpha (PPAR alpha), was subsequently shown to be activated by a variety of medium and long-chain fatty acids and to stimulate expression of the genes. The PPAR alpha binds to promoter domain of key enzymes concerning in the lipid catabolism system such as acyl-CoA synthase existing in the cytosol, acyl-CoA dehydrogenase and HMG-CoA synthase existing in the mitochondria and acyl-CoA oxidase existing in the peroxisome of liver. From the analysis of PPAR alpha-deficient mice, it is being considered that the PPAR alpha plays an important role for the energy acquisition in starvation state, that is, oxidation of fatty acid and formation of ketone body in liver.

Since the discovery of PPAR alpha additional isoforms of PPAR have been identified, PPAR beta, PPAR gamma and PPAR delta, which are spatially differentially expressed.

The nuclear peroxisome proliferator-activated receptor gamma (PPARgamma) activates the transcription of multiple genes involved in intra- and extracellular lipid metabolism. These PPARs regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis.

Because there are several isoforms of PPAR, it is desirable to identify compounds which are capable of selectively interacting with only one of the PPAR isoforms. Hypolipidaemic agents have the ability to stimulate PPAR alpha and the ensuing stimulation of peroxisomal proliferation and consequent fatty acid oxidation can account for the reduction in plasma fatty acids. PPAR-gamma plays a key role in adipocyte differentiation and insulin sensitivity—its selective synthetic ligands, the thiazolidinediones (TZD), are used as insulin sensitizers in the treatment of type 2 diabetes. Compounds also exist which exhibit agonist activity at both PPAR alpha and PPAR gamma and would be particularly effective for the treatment of obesity as well as for the treatment of diabetes/pre-diabetic insulin resistance syndrome and the resulting complications thereof. Function of PPAR delta is not very understood compared with alpha form or gamma form.

Knowledge of the mechanisms that regulate PDC activity is important, because PDC inactivation is crucial for glucose conservation when glucose is scarce, whereas adequate PDC activity is required to allow both ATP and FA production from glucose. Fuel metabolism is highly regulated to ensure adequate energy for cellular function. The contribution of the major metabolic fuels—glucose, lactate and fatty acids (FAs)—often reflects their circulating levels. In addition, regulatory cross-talk and fuel-induced hormone secretion ensures appropriate and co-ordinate fuel utilization.

Because its activity can either determine or reflect fuel preference (carbohydrate versus fat), the pyruvate dehydrogenase complex (PDC) occupies a pivotal position in fuel cross-talk. Active PDC permits glucose oxidation and allows the formation of mitochondrially-derived intermediates (e.g. malonyl-CoA and citrate) that reflect fuel abundance. FA oxidation suppresses PDC activity. PDC inactivation by phosphorylation is catalysed by pyruvate dehydrogenase kinases (PDKs) 1-4, which are regulated differentially by metabolite effectors. Most tissues contain at least two and often three of the PDK isoforms.

A hypothesis was developed that PDK4 is a “lipid status”—responsive PDK isoform facilitating FA oxidation and signalling through citrate formation. Substrate interactions at the level of gene transcription extend glucose-FA interactions to the longer term. Isoform-specific differences in kinetic parameters, regulation, and phosphorylation site specificity of the PDKs introduce variations in the regulation of PDC activity in differing endocrine and metabolic states. Thus potential targets for substrate-mediated transcriptional regulation in relation to selective PDK isoform expression and the influence of altered PDK isoform expression in fuel sensing, selection and utilization.

Adequate flux through PDC is important in tissues with a high ATP requirement, in lipogenic tissues (since it provides cytosolic acetyl-CoA for fatty acid (FA) synthesis), and in generating cytosolic malonyl-CoA, a potent inhibitor of carnitine palmitoyltransferase (CPT I). Conversely, suppression of PDC activity is crucial for glucose conservation when glucose is scarce. Recent advances relating to the control of mammalian PDC activity by phosphorylation (inactivation) and dephosphorylation (activation, reactivation), in particular regulation of PDC by pyruvate dehydrogenase kinase (PDK), which phosphorylates and inactivates PDC.

Inactivation of PDC by increased PDK activity promotes gluconeogenesis by conserving three-carbon substrates. PDK activity is that of a family of four proteins (PDK1-4). PDK2 and PDK4 appear to be expressed in most major tissues and organs of the body, PDK1 appears to be limited to the heart and pancreatic islets, and PDK3 is limited to the kidney, brain and testis. PDK4 is selectively upregulated in the longer term in most tissues and organs in response to starvation and hormonal imbalances such as insulin resistance, diabetes mellitus and hyperthyroidism. Parallel increases in PDK2 and PDK4 expression appear to be restricted to gluconceogenesic tissues, liver and kidney, which take up as well as generate pyruvate.

Immunoblot analysis with antibodies raised against recombinant PDK isoforms demonstrated changes in PDK isoform expression in response to experimental hyperthyroidism (100 microg/100 g body weight; 3 days) that was selective for fast-twitch vs slow-twitch skeletal muscle in that PDK2 expression was increased in the fast-twitch skeletal muscle (the anterior tibialis) (by 1. 6-fold; P<0.05) but not in the slow-twitch muscle (the soleus). PDK4 protein expression was increased by experimental hyperthyroidism in both muscle types, there being a greater response in the anterior tibialis (4.2-fold increase; P<0.05) than in the soleus (3.2-fold increase; P<0.05). The hyperthyroidism-associated up-regulation of PDK4 expression was observed in conjunction with suppression of skeletal-muscle PDC activity, but not suppression of glucose uptake/phosphorylation, as measured in vivo in conscious unrestrained rats (using the 2-[(3)H]deoxyglucose technique).

It was proposed that increased PDK isoform expression contributes to the pathology of hyperthyroidism and to PDC inactivation by facilitating the operation of the glucose-->lactate-->glucose (Cori) and glucose-->alanine-->glucose cycles. We also propose that enhanced relative expression of the pyruvate-insensitive PDK isoform (PDK4) in skeletal muscle in hyperthyroidism uncouples glycolytic flux from pyruvate oxidation, sparing pyruvate for non-oxidative entry into the tricarboxylic acid (TCA) cycle, and thereby supporting entry of acetyl-CoA (derived from fatty acid oxidation) into the TCA cycle.

Regulation of PDC determines and reflects substrate preference and is critical to the ‘glucose-fatty acid cycle’, a concept of reciprocal regulation of lipid and glucose oxidation to maintain glucose homoeostasis. Mammalian PDC activity is inactivated by phosphorylation by the PDKs (pyruvate dehydrogenase kinases). PDK inhibition by pyruvate facilitates PDC activation, favouring glucose oxidation and malonyl-CoA formation: the latter suppresses LCFA (long-chain fatty acid) oxidation. PDK activation by the high mitochondrial acetyl-CoA/CoA and NADH/NAD(+) concentration ratios that reflect high rates of LCFA oxidation causes blockade of glucose oxidation. Complementing glucose homoeostasis in health, fuel allostasis, i.e. adaptation to maintain homoeostasis, is an essential component of the response to chronic changes in glycaemia and lipidaemia in insulin resistance.

The concept that the PDKs act as tissue homoeostats, suggests that long-term modulation of expression of individual PDKs, particularly PDK4, is an essential component of allostasis to maintain homoeostasis. This also describes the intracellular signals that govern the expression of the various PDK isoforms, including the roles of the peroxisome proliferator-acivated receptors and lipids, as effectors within the context of allostasis.

Agonists of peroxisome proliferator-activated receptors (PPARs) have emerged as important pharmacological agents for improving insulin action. A major mechanism of action of PPAR agonists is thought to involve the alteration of the tissue distribution of nonesterified fatty acid (NEFA) uptake and utilization.

To test this hypothesis directly, the effect of the novel PPARa/g agonist tesaglitazar was examined on whole-body insulin sensitivity and NEFA clearance into epididymal white adipose tissue (WAT), red gastrocnemius muscle, and liver in rats with dietary-induced insulin resistance. Wistar rats were fed a high-fat diet (59 of calories as fat) for 3 wk with or without treatment with tesaglitazar (1 mmol.kg-1.d-1, 7 d). NEFA clearance was measured using the partially metabolizable NEFA tracer, 3H-R-bromopalmitate, administered under conditions of basal or elevated NEFA availability. Tesaglitazar improved the insulin sensitivity of high-fat-fed rats, indicated by an increase in the glucose infusion rate during hyperinsulinemic-euglycemic clamp (P<0.01). This improvement in insulin action was associated with decreased diglyceride (P<0.05) and long chain acyl coenzyme A (P<0.05) in skeletal muscle. NEFA clearance into WAT of high-fat-fed rats was increased 52 by tesaglitazar under basal conditions (P<0.001). In addition the PPARa/g agonist moderately increased hepatic and muscle NEFA utilization and reduced hepatic triglyceride accumulation (P<0.05).

This study shows that tesaglitazar is an effective insulin-sensitizing agent in a mild dietary model of insulin resistance. Furthermore, we provide the first direct in vivo evidence that an agonist of both PPARa and PPARg increases the ability of WAT, liver, and skeletal muscle to use fatty acids in association with its beneficial effects on insulin action in this model.

Liver contains two pyruvate dehydrogenase kinases (PDKs), namely PDK2 and PDK4, which regulate glucose oxidation through inhibitory phosphorylation of the pyruvate dehydrogenase complex (PDC). Starvation increases hepatic PDK2 and PDK4 protein expression, the latter occurring, in part, via a mechanism involving peroxisome proliferator-activated receptor-alpha (PPARalpha). High-fat feeding and hyperthyroidism, which increase circulating lipid supply, enhance hepatic PDK2 protein expression, but these increases are insufficient to account for observed increases in hepatic PDK activity. Enhanced expression of PDK4, but not PDK2, occurs in part via a mechanism involving PPAR-alpha.

Fatty acid metabolism is transcriptionally regulated by two reciprocal systems: peroxisome proliferator-activated receptor (PPAR) a controls fatty acid degradation, whereas sterol regulatory element-binding protein-1c activated by liver X receptor (LXR) regulates fatty acid synthesis. To explore potential interactions between LXR and PPAR, the effect of LXR activation on PPARa signaling was investigated. In luciferase reporter gene assays, overexpression of LXRa or b suppressed PPARa-induced peroxisome proliferator response element-luciferase activity in a dose-dependent manner. LXR agonists, T0901317 and 22(R)-hydroxycholesterol, dose dependently enhanced the suppressive effects of LXRs. Gel shift assays demonstrated that LXR reduced binding of PPARa/retinoid X receptor (RXR) a to peroxisome proliferator response element.

Addition of increasing amounts of RXRa restored these inhibitory effects in both luciferase and gel shift assays, suggesting the presence of RXRa competition. In vitro protein binding assays demonstrated that activation of LXR by an LXR agonist promoted formation of LXR/RXRa and, more importantly, LXR/PPARa heterodimers, leading to a reduction of PPARa/RXRa formation.

Supportively, in vivo administration of the LXR ligand to mice and rat primary hepatocytes substantially decreased hepatic mRNA levels of PPARa-targeted genes in both basal and PPARa agonist-induced conditions. The amount of nuclear PPARa/RXR heterodimers in the mouse livers was induced by treatment with PPARa ligand, and was suppressed by superimposed LXR ligand. Taken together with data from the paper (Yoshikawa, T., T. Ide, H. Shimano, N. Yahagi, M. Amemiya-Kudo, T. Matsuzaka, S. Yatoh, T. Kitamine, H. Okazaki, Y. Tamura, M. Sekiya, A. Takahashi, A. H. Hasty, R. Sato, H. Sone, J. Osuga, S. Ishibashi, and N. Yamada, Endocrinology 144:1240-1254) describing PPARa suppression of the LXR-sterol regulatory element-binding protein-1c pathway, it has been proposed that the presence of an intricate network of nutritional transcription factors with mutual interactions, resulting in efficient reciprocal regulation of lipid degradation and lipogenesis.

Heterodimerization partners for retinoid X receptors (RXRs) include PPARalpha and thyroid-hormone receptors (TRs). The responses were investigated of hepatic PDK protein expression to high-fat feeding and hyperthyroidism in relation to hepatic lipid delivery and disposal. High-fat feeding increased hepatic PDK2, but not PDK4, protein expression whereas hyperthyroidism increased both hepatic PDK2 and PDK4 protein expression. Both manipulations decreased the sensitivity of hepatic carnitine palmitoyltransferase I (CPT I) to suppression by malonyl-CoA, but only hyperthyrodism elevated plasma fatty acid and ketone-body concentrations and CPT I maximal activity.

Administration of the selective PPAR-alpha activator WY14,643 significantly increased PDK4 protein to a similar extent in both control and high-fat-fed rats, but WY14,643 treatment and hyperthyroidism did not have additive effects on hepatic PDK4 protein expression. PPARalpha activation did not influence hepatic PDK2 protein expression in euthyroid rats, suggesting that up-regulation of PDK2 by hyperthyroidism does not involve PPARalpha, but attenuated the effect of hyperthyroidism to increase hepatic PDK2 expression. The results indicate that hepatic PDK4 up-regulation can be achieved by heterodimerization of either PPAR alpha or TR with the RXR receptor and that effects of PPAR alpha activation on hepatic PDK2 and PDK4 expression favour a switch towards preferential expression of PDK4.

The pyruvate dehydrogenase complex (PDC) occupies a strategic role in renal intermediary metabolism, via partitioning of pyruvate flux between oxidation and entry into the gluconeogenic pathway. Inactivation of PDC via activation of pyruvate dehydrogenase kinases (PDKs), which catalyze PDC phosphorylation, occurs secondary to increased fatty acid oxidation (FAO). In kidney, inactivation of PDC after prolonged starvation is mediated by up-regulation of the protein expression of two PDK isoforms, PDK2 and PDK4. The lipid-activated transcription factor, peroxisome proliferator-activated receptor-alpha (PPAR alpha), plays a pivotal role in the cellular metabolic response to fatty acids and is abundant in kidney.

In the present study PPAR alpha null mice were used to examine the potential role of PPAR alpha in regulating renal PDK protein expression. In wild-type mice, fasting (24 h) induced marked up-regulation of the protein expression of PDK4, together with modest up-regulation of PDK2 protein expression. In striking contrast, renal protein expression of PDK4 was only marginally induced by fasting in PPAR alpha null mice. The present results define a critical role for PPAR alpha in renal adaptation to fasting, and identify PDK4 as a downstream target of PPAR alpha activation in the kidney.

It has been proposed that specific up-regulation of renal PDK4 protein expression in starvation, by maintaining PDC activity relatively low, facilitates pyruvate carboxylation to oxaloacetate and therefore entry of acetyl-CoA derived from FA beta-oxidation into the TCA cycle, allowing adequate ATP production for brisk rates of gluconeogenesis.

Factors that regulate PDK4 expression include FA oxidation and adequate insulin action. PDK4 is also either a direct or indirect target of peroxisome proliferator-activated receptor (PPAR) alpha. PPAR alpha deficiency in liver and kidney restricts starvation-induced upregulation of PDK4; however, the role of PPAR alpha in heart and skeletal muscle appears to be more complex. These observations may have important implications for the pharmacological modulation of PDK activity (e.g. use of PPAR alpha activators) for the control of whole-body glucose, lipid and lactate homeostasis in disease states and suggest that therapeutic interventions must be tissue targeted so that whole-body fuel homeostasis is not adversely perturbed.

Regulation of the activity of the pyruvate dehydrogenase complex in skeletal muscle plays an important role in fuel selection and glucose homeostasis. Activation of the complex promotes disposal of glucose, whereas inactivation conserves substrates for hepatic glucose production. Starvation and diabetes induce a stable increase in pyruvate dehydrogenase kinase activity in skeletal muscle mitochondria that promotes phosphorylation and inactivation of the complex.

The present study shows that these metabolic conditions induce a large increase in the expression of PDK4, one of four pyruvate dehydrogenase kinase isoenzymes expressed in mammalian tissues, in the mitochondria of gastrocnemius muscle. Refeeding starved rats and insulin treatment of diabetic rats decreased pyruvate dehydrogenase kinase activity and also reversed the increase in PDK4 protein in gastrocnemius muscle mitochondria. Starvation and diabetes also increased the abundance of PDK4 mRNA in gastrocnemius muscle, and refeeding and insulin treatment again reversed the effects of starvation and diabetes.

These findings suggest that an increase in amount of this enzyme contributes to hyperphosphorylation and inactivation of the pyruvate dehydrogenase complex in these metabolic conditions. It was further found that feeding rats WY-14,643, a selective agonist for the peroxisome proliferator-activated receptor-alpha (PPAR-alpha), also induced large increases in pyruvate dehydrogenase kinase activity, PDK4 protein, and PDK4 mRNA in gastrocnemius muscle. Since long-chain fatty acids activate PPAR-alpha endogenously, increased levels of these compounds in starvation and diabetes may signal increased expression of PDK4 in skeletal muscle.

The transcriptional coactivator PPAR gamma coactivator 1 alpha (PGC-1 alpha) is a key regulator of metabolic processes such as mitochondrial biogenesis and respiration in muscle and gluconeogenesis in liver. Reduced levels of PGC-1 alpha in humans have been associated with type II diabetes. PGC-1 alpha contains a negative regulatory domain that attenuates its transcriptional activity. This negative regulation is removed by phosphorylation of PGC-1 alpha by p38 MAPK, an important kinase downstream of cytokine signaling in muscle and beta-adrenergic signaling in brown fat.

Described here the identification of p160 myb binding protein (p160 MBP) as a repressor of PGC-1 alpha. The binding and repression of PGC-1alpha by p160 MBP is disrupted by p38 MAPK phosphorylation of PGC-1 alpha. Adenoviral expression of p160 MBP in myoblasts strongly reduces PGC-1alpha's ability to stimulate mitochondrial respiration and the expression of the genes of the electron transport system. This repression does not require removal of PGC-1 alpha from chromatin, suggesting that p160 MBP is or recruits a direct transcriptional suppressor. Overall, these data indicate that p160 MBP is a powerful negative regulator of PGC-1 alpha function and provide a molecular mechanism for the activation of PGC-1 alpha by p38 MAPK.

In rat pancreatic islets chronically exposed to high glucose or high free fatty acid (FFA) levels, glucose-induced insulin release and mitochondrial glucose oxidation are impaired. These abnormalities are associated with high basal ATP levels but a decreased glucose-induced ATP production (Delta of increment over baseline 0.7+/−0.5 or 0.5+/−0.3 pmol/islet in islets exposed to glucose or FFA vs. 1 2.0+/−0.6 in control islets, n=3; P<0.01) and, as a consequence, with an altered ATP/ADP ratio.

To investigate further the mechanism of the impaired ATP formation, in rat pancreatic islets glucose-stimulated pyruvate dehydrogenase (PDH) activity was measured, a key enzyme for pyruvate metabolism and for the subsequent glucose oxidation through the Krebs cycle, and also the uncoupling protein-2 (UCP-2) content by Western blot. In islets exposed to high glucose or FFA, glucose-stimulated PDH activity was impaired and UCP-2 was overexpressed. Because UCP-2 expression is modulated by a peroxisome proliferator-activated receptor (PPAR)-dependent pathway, PPAR-gamma contents were measured by Western blot and the effects of a PPAR-gamma antagonist. PPAR-gamma levels were overexpressed in islets cultured with high FFA levels but unaffected in islets exposed to high glucose. In islets exposed to high FFA concentration, a PPAR-gamma antagonist was able to prevent UCP-2 overexpression and to restore insulin secretion and the ATP/ADP ratio.

These data indicate that in rat pancreatic islets chronically exposed to high glucose or FFA, glucose-induced impairment of insulin secretion is associated with (and might be due to) altered mitochondrial function, which results in impaired glucose oxidation, overexpression of the UCP-2 protein, and a consequent decrease of ATP production. This alteration in FFA cultured islets is mediated by the PPAR-gamma pathway.

Methyl pyruvate has been described with reference to a particular embodiment. For one skilled in the art, other modifications and enhancements can be made without departing from the spirit and scope of the aforementioned claims.

Whilst endeavoring in the foregoing Specification to draw attention to those features of the invention believed to be of particular importance it should be understood that the Applicant claims protection in respect of any patentable feature hereinbefore referred to whether or not particular emphasis has been placed thereon.

What is claimed is:

1. A method of increasing cellular energy production with the use of methyl pyruvate in a human.

2. A method of increasing cellular energy production with the use of methyl pyruvic acid in a human.

3. A method of increasing methyl pyruvate levels and said effects in a human.

4. A method of increasing methyl pyruvic acid levels and said effects in a human.

5. The method of claim 2 wherein a therapeutic and effective amount of methyl pyruvic acid is infused or orally administered to the human.

6. The method of claim 1 wherein a therapeutic and effective amount of the salt of methyl pyruvate is infused or orally administered to the human.

7. The method of claim 6 wherein the salt of methyl pyruvate is a monovalent cation (such as sodium or potassium methyl pyruvate).

8. The method of claim 6 wherein the salt of methyl pyruvate is a divalent cation (such as calcium or magnesium methyl pyruvate).

9. The method of claim 6 wherein analogs of these compounds can act as substrates or substrate analogs for methyl pyruvate.

10. The method of claim 6 wherein the salt of methyl pyruvate and composition of a pharmacologically acceptable excipient and/or diluent therefore.

11. The method of claim 10 wherein the salt of methyl pyruvate and composition which further may comprise vitamins, coenzymes, mineral substances, amino acids, herbs and antioxidants or pharmaceutical drugs.

12. The method of claim 10, infused or orally administrable, in the form of a dietary supplement, energizer or pharmaceutical drug.

13. The method of claim 11, infused or orally administrable, in the form of a dietary supplement, energizer or pharmaceutical drug.

14. The method of claim 12, in the form of lozenges, tablets, pills, capsules, powders, granulates, sachets, syrups or vials.

15. The method of claim 13, in the form of lozenges, tablets, pills, capsules, powders, granulates, sachets, syrups or vials.

16. The method of claim 14, in unit dosage form, comprising from about 100 mg to about 28 grams.

17. The method of claim 15, in unit dosage form, comprising from about 100 mg to about 28 grams.

18. The method of claim 17, for treating a subject afflicted with a viral infection comprising administering to the subject an amount of methyl pyruvate salt, such that the subject is treated for a viral infection.

19. The method of claim 17, for treating a subject for the negative side-effects of viral infection treatment who is afflicted with and being treated for a viral infection, comprising administering to the subject an amount of methyl pyruvate salt, such that the subject is treated for viral infection treatment negative side-effects.

20. The method of claim 5, wherein methyl pyruvic acid and composition of a pharmacologically acceptable excipient and/or diluent therefore.

21. The method of claim 20, wherein methyl pyruvic acid and composition which further may comprise vitamins, coenzymes, mineral substances, amino acids, herbs and antioxidants or pharmaceutical drugs.

22. The method of claim 20, infused or orally administrable, in the form of a dietary supplement, energizer or pharmaceutical drug.

23. The method of claim 21, infused or orally administrable, in the form of a dietary supplement, energizer or pharmaceutical drug.

24. The method of claim 22, in the form of lozenges, tablets, pills, capsules, powders, granulates, sachets, syrups or vials.

25. The method of claim 23, in the form of lozenges, tablets, pills, capsules, powders, granulates, sachets, syrups or vials.

26. The method of claim 24, in unit dosage form, comprising from about 100 mg to about 28 grams.

27. The method of claim 25, in unit dosage form, comprising from about 100 mg to about 28 grams.

28. The method of claim 27, for treating a subject afflicted with a viral infection comprising administering to the subject an amount of methyl pyruvic acid, such that the subject is treated for a viral infection.

29. The method of claim 27, for treating a subject for the negative side-effects of viral infection treatment who is afflicted with and being treated for a viral infection, comprising administering to the subject an amount of methyl pyruvic acid, such that the subject is treated for viral infection treatment negative side-effects.

30. The method of claim 17, in supporting PARP-1 activation for ensuring genomic stability and ablation of viral infection, also including all ameliorating effects of said support, comprising the step of administering to a human at risk a therapeutically effective quantity of said substance to cells to promote ATP/NAD metabolism.

31. The method of claim 27, in supporting PARP-1 activation for ensuring genomic stability and ablation of viral infection, also including all ameliorating effects of said support, comprising the step of administering to a human at risk a therapeutically effective quantity of said substance to cells to promote ATP/NAD metabolism.

32. The method of claim 17, in promoting PPAR up-regulation and all ameliorating effects of said up-regulation, comprising the step of administering to a human at risk a therapeutically effective quantity of said substance.

33. The method of claim 27, in promoting PPAR up-regulation and all ameliorating effects of said up-regulation, comprising the step of administering to a human at risk a therapeutically effective quantity of said substance.

34. The method of claim 30, for protecting a human cell against death, impairment or degeneration induced by ATP/NAD depletion triggered by PARP-1 activation from an ischemic event, comprising the step of injecting, into the bloodstream of a human at risk of ischemic damage, a therapeutically effective quantity.

35. The method of claim 31, for protecting a human cell against death, impairment or degeneration induced by ATP/NAD depletion triggered by PARP-1 activation from an ischemic event, comprising the step of injecting, into the bloodstream of a human at risk of ischemic damage, a therapeutically effective quantity.

36. The method of claim 34, wherein administered to the human in conjunction with insulin.

37. The method of claim 35, wherein administered to the human in conjunction with insulin.
